(19) United States
(12) Patent Application Publication
(10) Pub. No.: US 2021/0355221 A1
(43) Pub. Date: Nov. 18, 2021

(54) Targeting the Non-Canonical NFkB Pathway in Cancer Immunotherapy

(71) Applicant: The General Hospital Corporation, Boston, MA (US)

(72) Inventors: Mikael J. Pittet, Charlestown, MA (US); Ralph Weissleder, Peabody, MA (US); Christopher Garris, Wellesley, MA (US); Sean Arlauckas, Boston, MA (US)

(21) Appl. No.: 17/284,946

(22) PCT Filed: Oct. 18, 2019
(86) PCT No.: PCT/US2019/057057

§ 371 Date: Apr. 13, 2021

Related U.S. Application Data
(60) Provisional application No. 62/747,406, filed on Oct. 18, 2018.

(51) Int. Cl. C07K 16/28; A61K 45/06; A61P 35/00

(52) U.S. Cl. CPC C07K 16/2818; C07K 16/2827; A61K 2039/505; A61P 35/00; A61K 45/06

(57) ABSTRACT

Methods of treating a subject (e.g., a mammalian, preferably human, subject) with cancer, e.g., with melanoma, comprising administering a combination of an inhibitor of the non-canonical NFkB pathway and a checkpoint inhibitor.

### CLAIM OF PRIORITY

This application claims the benefit of U.S. Provisional Patent Application Ser. No. 62/747,406, filed on Oct. 18, 2018. The entire contents of the foregoing are hereby incorporated by reference.

### BACKGROUND

Immune checkpoint blockade has emerged as a critical treatment against various cancer types (Topalian et al., 2012). Currently approved immune checkpoint blockers are monoclonal antibodies that target the cytotoxic T lymphocyte-associated protein 4 (CTLA-4) or programmed cell death protein 1 (PD-1) pathways. These inhibitory pathways are important because they protect the host from uncontrolled immune activation (Keir et al., 2008) but they can also be co-opted by tumors, which make them resist immune attack (Wherry, 2011). For instance, tumor-infiltrating cytotoxic CD8+ T cells often express PD-1 that renders them ineffective against tumors. Consequently, anti-PD-1 (aPD-1) mAbs, or anti-PDL1 mAbs, are designed to antagonize the PD-1 inhibitory pathway in T cells and potentiate CD8+ T cell-mediated tumor destruction.

### SUMMARY

The disclosure relates to the discovery from real-time in vivo imaging studies and single cell RNA sequencing that anti-tumor dendritic cells are enriched for components of the non-canonical NFkB signaling pathway. Agonizing the non-canonical NFkB pathway therapeutically can enhance anti-cancer immunity. Targeting the non-canonical NFkB pathway can be used for anti-cancer therapeutics.

Anti-PD-1 immune checkpoint blockers can induce sustained clinical responses in cancer but how they function in vivo remains incompletely understood. Here, we combined intravital real-time imaging with single cell RNA sequencing analysis and mouse models to uncover anti-PD-1 pharmacodynamics directly within tumors. We showed that effective antitumor responses required a subset of tumor-infiltrating dendritic cells (DCs), which produced interleukin 12 (IL-12). These DCs did not bind anti-PD-1 but produced IL-12 upon sensing interferon γ (IFN-γ) that was released from neighboring T cells. In turn, DC-derived IL-12 stimulated antitumor T cell immunity. These findings suggest that full-fledged activation of antitumor T cells by anti-PD-1 is not direct, but rather involves T cell:DC crosstalk and is licensed by IFN-γ and IL-12. Furthermore, we found that activating the non-canonical NFkB transcription factor pathway amplified IL-12-producing DCs and sensitized tumors to anti-PD-1 treatment, suggesting a therapeutic strategy to improve responses to checkpoint blockade.

Thus, provided herein are methods for treating a subject (e.g., a mammal, e.g., a human) with cancer. The methods include administering an inhibitor of the non-canonical NFkB pathway and a checkpoint inhibitor. In some embodiments, the subject has melanoma. Also provided herein are a composition comprising an inhibitor of the non-canonical NFkB pathway and a composition comprising a checkpoint inhibitor for use in a method of treating a subject with cancer.

In some embodiments, the checkpoint inhibitor is an antibody, e.g., anti-PD1 or anti-PDL1.

In some embodiments, the inhibitor of the non-canonical NFkB pathway is a NIK inhibitor. In some embodiments, the NIK inhibitor is selected from the group consisting of alkynyl alcohols; 6-membered heteroaromatic substituted cyanoindoline derivatives; pyrazoloisoquinoline derivatives; 6-azaindole aminopyrimidine derivatives; pyrazoloisoquinoline derivatives; sulfapyridine; propranolol; tricyclic NF-κB inducing kinase inhibitors; 4H-isoquinoline-1,3-dione and 2,7-naphthydrine-1,3,6,8-tetrone; N-Acetyl-3-aminopyrazoles; NIK-SMI1 ((R)-6-(3-((3-hydroxy-1-methyl-2-oxopyrrolidin-3-yl)ethynyl)phenyl)-4-methoxypicolinamide), AM-0216 ((R)-4-(1-(2-aminopyrimidin-4-yl)indolin-6-yl)-2-(thiazol-2-yl)but-3-yn-2-01), AM-0561 ((R)-4-(3-(2-amino-5-chloropyrimidin-4-yl)imidazo[1,2-a]pyridin-6-yl)-2-(thiazol-2-yl)but-3-yn-2-ol), or Amgen16 (1-((1-(2-amino-5-chloropyrimidin-4-yl)indolin-6-yl)ethynyl)cyclopentan-1-01).

In some embodiments, the inhibitor of the non-canonical NFkB pathway and the checkpoint inhibitor are in, or are administered in, a single composition.

In some embodiments, in addition to or as an alternative to the inhibitor of the non-canonical NFkB pathway, the methods can include targeted intratumoral delivery of IL-12 encoding plasmids (e.g., as described in Daud et al., 2008) in combination with an immunotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. Successful aPD-1 treatment triggers endogenous IFN-γ and IL-12 responses within tumors. (A) Diagram describing intravital imaging of MC38-H2B-mApple tumors implanted in cytokine-reporter mice for tracking lymphoid and myeloid cell pharmacodynamics (PD) after aPD-1 treatment. (B) Left: Intravital micrographs of MC38 tumors in IFN-γ-eYFP reporter mice treated or not with aPD-1 mAb (n=3 mice/group). IFN-γ-eYFP expressing cells; tumor cells; and PacificBlueFMX-labeled tumor-associated macrophages (TAM) are shown. Right: Fold change of IFN-γ⁺ cells in both groups at different times after treatment and compared to baseline. (C) Same as in (B) but in IL-12p40-eYFP reporter mice (n=5 mice/group). (D) Representative intravital micrographs of H2B-mApple MC38 tumor edge or core obtained in IL-12p40 reporter mice before (left), one day after (middle) and 5 days after (right) aPD-1 treatment. PacBlue-labeled dextran was used to locate tumor vessels. Scale bars represent 30 μm. (E) Distance between IL-12p40⁺ cells and the tumor margin measured by intravital imaging. Each point represents a single cell (n=8 control and 5 aPD-1-treated mice). (F) Distance between IL-12p40⁺ cells and closest tumor vessel measured by intravital imaging. Each point represents a single cell (n=5 mice/group). (G) In vivo time-lapse microscopy of IL-12p40 reporter mice tracking IL-12⁺ cell motility after aPD-1 treatment. Track plots represent displacement from origin of IL-12⁺ cells in the tumor microenvironment. (H) Motility coefficient was calculated for each IL-12⁺ cell at both time points. n.s.=not significant, **p<0.01, ****p<0.0001. Values represent SEM. Data are representative of at least two independent experiments. For comparisons between two groups, Student's two-tailed t-test was used. See also FIG. 8.

FIGS. 2A-I. IL-12 is produced by DC1s and is necessary for treatment efficacy. (A) t-SNE plot using scRNAseq data from CD45⁺ cells sorted from MC38 tumors 3 days after aPD-1 treatment. Untreated mice served as control. Control and aPD-1 samples are pooled. (B-E) Violin plots showing the gene expression probability distribution of various dendritic cell markers (B), colony stimulating factor receptors (C), costimulation factors (D), and chemokine and chemokine receptors (E), in DC₁, DC₂ and other immune cell clusters (Mø, macrophages; Mo, monocytes; Neu, neutrophils; NK, natural killer cells; T_{conv}, conventional T cells; T_{reg}, regulatory T cells). (F) Feature plot of Il12b expression across cell clusters identified in A. (G) Expression in DC₁ and DC₂ of genes associated with IL-12 production. (H) MC38 tumor volumes in Zbtb46-DTR bone marrow chimeras treated or not with diphtheria toxin (DT) to deplete DCs prior to aPD-1 or control treatment. (I) MC38 tumor volume in mice treated with aPD-1 (black), aPD-1 and aIL-12, or vehicle (gray); n=15 mice/group. Data are representative of at least two independent experiments. Arrows indicate duration of treatment. n.s.=not significant, *p<0.05, ***p<0.001. Values represent SEM. For comparisons between three or more groups, One way ANOVA with multiple comparisons was used. See also FIG. 9.

FIGS. 3A-F. DC-mediated IL-12 production requires IFN-γ sensing. (A) Flow cytometry measurement of PD-1 expression across cell types in the MC38 tumor microenvironment. (B) Intravital micrographs of the MC38 tumor microenvironment in an IL12 reporter mouse five days after AF647-aPD-1 treatment. Tumor cells, TAM, IL-12p40, aPD-1 are shown. (C) Intravital micrographs and quantification of IL-12p40 signal two days after aPD-1 treatment in the tumor microenvironment after CD8 depletion. Tumor cells and IL-12p40 are shown. Data plotted as fold change in IL-12p40 from baseline levels. (D) MC38 tumors were harvested at 3 days post-treatment with aPD-1 in combination with aIFN-γ or control, and processed for RNA isolation. Quantitative PCR for IL12p40 gene expression data are normalized with control sample values set to 1. (E) Relative IL-12p40 gene expression in MC38 tumors from CD11c-cre (Itgax-cre)×IFNγR1^{fl/fl} (IFNγR-deficient) or control (IFNγR1^{fl/fl}) mice three days after aPD-1 treatment. (F) Change in MC38 tumor volume on day six after aPD-1 treatment in IFNγR-deficient or control mice. Data are relative to pre-treatment tumor volumes. Data are representative of at least two independent experiments. n. s.=not significant, *p<0.05, **p<0.01, ***p<0.001, ****p<0.0001. For comparisons between two groups, Student's two-tailed t-test was used. For comparisons between three or more groups, One way ANOVA with multiple comparisons was used. See also FIG. 10.

FIG. 4AC-. IL-12 activates TILs directly in mice. (A) Left: Intravital micrographs of MC38 tumors in IFN-γ-eYFP reporter mice before or four days after treatment with recombinant IL-12. IFN-γ-eYFP expressing cells; MC38 tumor cells. Right: Fold change of IFN-γ⁺ cells in treated and untreated groups compared to baseline. Arrow indicates duration of IL-12 treatment. (B) MC38 tumor growth monitored after mice bearing established tumors were treated with recombinant IL-12 or control for 5 days; n≥3 per group. (C) Tumor-infiltrating CD8⁺ T cells isolated from MC38 tumors, stimulated in vitro with anti-CD3/CD28 and/or IL-12, and assessed by flow cytometry for intracellular IFN-γ production. Data show IFN-γ mean fluorescent intensity (MFI; n=3 per group). Data are representative of at least two independent experiments. **p<0.01, ***p<0.001, ****p<0.0001. For comparisons between two groups, Student's two-tailed t-test was used. For comparisons between three or more groups, One way ANOVA with multiple comparisons was used. See also FIG. 11.

FIGS. 5A-D. IL-12 activates TILs directly in cancer patients. (A) Relative expression levels of cytolytic signature genes measured by Nanostring in skin tumor biopsies from 19 melanoma patients both before (light gray dots) and after (dark gray dots) intratumoral treatment with ImmunoPulse IL-12. Data are normalized to pre-treatment biopsy expression levels; POL2RA is a control gene. (B) Heat map of individual patient gene expression from melanoma biopsies from (A). Cytolytic signature genes are displayed as fold change over pre-treatment levels for each individual patient. OAZ1, POLR2A, and SDHA are control genes. (C) Clinical outcomes data from patients receiving ImmunoPulse treatment. SD, stable disease; PR, partial response; PD, progressive disease. Cytolytic signature was calculated as the sum of total cytolytic gene signature expression from (B). Values were stratified by the top, middle, and bottom third, and then associated to patient response status. (D) IFN-γ production by tumor-infiltrating CD8⁺ T cells isolated from six cancer patients, stimulated ex vivo with aCD3 and/or IL-12, and measured by ELISA. n.s.=not significant, ND=not detected, *p<0.05, **p<0.01, ***p<0.001. For comparisons between two groups, Student's two-tailed t-test was used. See also FIG. 12.

FIGS. 6A-E. Molecular targeting of the non-canonical NFkB pathway stimulates IL12-producing DCs. (A) Expression of non-canonical NFkB pathway components (illustrated on the left) across immune populations. (B) Intravital micrographs of a MC38 tumor in an IL-12p40 reporter mouse treated with AF647-aCD40 mAbs. Tumor cells, AF647-aCD40, IL-12p40, and TAM are shown. Dashed line highlights the location of an IL-12p40⁺ cell; ∇ show TAM overlaying with aCD40 mAbs. (C) Left: Intravital micrographs of MC38 tumors in IL-12p40-eYFP reporter mice treated with aCD40 or AZD5582. Untreated mice were used as controls. IFN-γ-eYFP expressing cells and tumor cells are shown. Right: Fold change of IL-12p40⁺ cells in each group after 48 hours and compared to baseline. (D-E) Ex vivo flow cytometry analysis of MC38 tumors in IL-12p40 reporter mice treated or not 48 h prior with agonistic aCD40 mAbs. CD45⁺F4/80⁺ TAMs (black) and CD45⁺F4/80⁻CD11c^{hi} MHCII^{hi} DCs (grey). (D) Fold change of IL-12p40⁺ cells normalized to untreated mice (E) MFI of IL-12 reporter signal from TAM or DC. Data are representative of at least two independent experiments. n. s.=not significant, *p<0.05, **p<0.01, ***p<0.001, ****p<0.0001, One way ANOVA with multiple comparisons. See also FIG. 13.

FIGS. 7A-G. Amplification of IL12-producing DCs improves cancer immunotherapy in an IL-12-dependent manner. (A) Intravital images of MC38 tumors in IFN-γ reporter mice treated with control mAb (left image) or agonistic aCD40 mAb (right image). Images were recorded one day after treatment. MC38 tumor cells; tumor-associated macrophages (TAM); and IFN-γ-producing cells are shown. Scale bars represent 30 μm. Longitudinal imaging of control or aCD40-treated mice was used to quantitate the change in density of IFN-γ-expressing cells compared to pre-treatment (graph at bottom). For both mouse cohorts, at least 10 fields of view per time-point were used. (B) MC38 tumor volume change after aCD40 or AZD5582 treatment in MC38 tumor-bearing mice with or without neutralizing IL-12 mAbs (aIL-12). Data are normalized to pre-treatment tumor volumes for individual mice, n=7-9 mice/group. (C) Survival of MC38 tumor-bearing mice treated with aCD40, aPD-1 or aPD-1+aCD40. Untreated mice served as controls, n≥6 mice/group. (D) Survival of B16F10 melanoma tumor-bearing mice treated with aCD40, aPD-1 or aPD-1+aCD40. Untreated mice served as controls, n=7-12 mice/group. (E) Mice cured with aPD-1+aCD40 (see panel F) were re-challenged ˜50 d later with B16F10 melanoma cells. Naive mice challenged at the same time served as positive controls. Data show the percent of mice rejecting B16F10 tumor re-challenge in each group. (F) Change in B16F10 tumor volume following treatment with aPD-1, IL-12 or both. Untreated mice served as controls, n≥5 mice/group. (G) Change in B16F10 tumor volume following treatment with aCD40, aPD-1+aCD40 or aPD-1+aCD40+aIL-12. Untreated mice served as controls, n≥5 mice/group. Data are representative of at least two independent experiments. n. s.=not significant, *p<0.05, **p<0.01, ***p<0.001, ****p<0.0001. For comparisons between two groups, Student's two-tailed t-test was used. For comparisons between three or more groups, One way ANOVA with multiple comparisons was used. See also FIG. 14.

FIGS. 8A-E. Characterization of IFN-!+CD8+ T Cells and IL-12p40+ DCs After aPD-1 Therapy. (A) Quantification of IFN-! signal from intravital microscopy of IFN-! reporter mice treated or not with aPD-1 mAbs (n=3 mice/group). Cell counts are expressed as fold change of IFN-!+ cells/mm2 from pre-treatment baseline. (B) Flow cytometry of aPD-1-treated MC38 tumors from IFN-! reporter mice shows IFN-! expression by CD8α+ cells. Gating strategy for IFN-!+ cells is shown for an aPD-1 treated sample. (C) IL-12p40+ cells per mm2 were quantified using intravital microscopy of MC38 tumors in IL-12p40 reporter mice treated with and without aPD-1 treatment. Values were calculated as a fold change from pre-treatment baseline (n=5 mice/group). IL-12 and IL-23 share the p40 subunit but have contrasting roles in cancer immunity, with IL-12 as antitumor and IL-23 as pro-tumor (Yan et al., 2017). Our data indicate responses due to IL-12 biological activity considering the lack of detectable IL-23 production in this experimental setting (FIG. 9A) and association of IL-12p40 with an anti-tumor response. (D) IL-12p40 reporter mice bearing MC38 tumors were treated with aPD-1 and tumors were harvested 3 days after treatment. Single cell suspensions of the tumors were prepared and stained for flow cytometry. Shown are the following subsets cells (pre-gated on CD45+): MHCII+F4/80−, F4/80+ and MHCII−F4/80−. (E) Congenic CD45.3 and IL-12p40 reporter mice were parabiosed and implanted with MC38 tumors. Mice were then treated with aPD-1 and tumors were isolated for flow cytometry analysis of IL-12-producing cells. Data are representative of 3 parabiotic mouse pairings. **p-value<0.01, Student's t-test two tailed.

FIGS. 9A-E. Characterization of scRNA Sequencing of MC38 Tumor Immune Infiltrates. (A) t-stochastic neighbor embedding (t-SNE) feature plots are clustered according to cell lineage defining factors, and assigned to immune cell types. Examples of defining factors are enumerated, and correspond to NK populations, Ncr1 and Klrb1c; Neutrophil populations, Cxcr2 and G0s2; T regulatory cells, Foxp3; T conventional cells, Cd3e, Cd8a, Pdcd1 and Ifng; Dendritic Cells, Zbtb46, Batf3 and Fscn1, and Monocytes/Macrophages, Lyz2 and Csf1r. Il23a is shown as control. DC, dendritic cell; Mø, macrophage; Mo, monocyte; Neu, neutrophil; NK, natural killer cell; Tconv, conventional T cell; Treg, regulatory T cell. (B) SPRING plots of selected cluster defining transcripts. Neutrophils, Cxcr2; NK cells, Ncr1; CD8+ T cells, Cd8a; T regulatory cells, Foxp3; Macrophages and monocytes, Csf1r; DC1, Il12b; DC2, Cd209a. Grey dots identify cells expressing each respective factor. DC, dendritic cell; Mø, macrophage; Mo, monocyte; Neu, neutrophil; NK, natural killer cell; Tconv, conventional T cell; Treg, regulatory T cell. (C) Itgae (Cd103) expression in DC1 cells identified by SPRING analysis either in control (left) or aPD-1 treated (right) animals. (D) IL-12p40 reporter mice were injected i.v. with B16F10 cells and lungs were processed for flow cytometry after 10 days of tumor growth. DCs were separated into CD103+CD11b− and CD103−CD11b+ subsets. Histograms show IL-12p40 expression in these subsets. Plots are representative of 5 mice. (E) Same as in (C) but for Il12b expression.

FIGS. 10A-G aPD-1 Induces IL-12 Production Indirectly through IFN-! Signaling. (A) The expression pattern of selected murine Fc receptors across immune cells clustered using SPRING analysis of MC38 tumor immune infiltrates analyzed by scRNA seq. (B) H2B-mApple MC38 tumor-bearing IL-12p40 reporter mice were treated with AlexaFluor647-aPD-1 mAbs and analyzed by intravital imaging. The data show the percent of aPD-1 signal overlapping with IL-12p40+ cells or with tumor-associated macrophages (TAMs) 24 h after aPD-1 administration. (C) AF647-aPD-1 mAb was administered to IL-12p40 reporter mice bearing H2B− mApple MC38 tumors and in vivo microscopy images above represent drug distribution within the first hour of administration. MC38 tumor cells; tumor associated macrophages (TAM); IL-12p40+ cells; and AF647-aPD-1 mAb are shown. Scale bars represent 30 μm. (D) Flow cytometry measurement of IL-12p40 signal (MFI, mean fluorescent intensity) in MC38 tumors three days after aPD-1 treatment and in the presence or absence of IFN-! neutralizing mAbs (aIFN-!). Data normalized to baseline IL-12p40 levels from n=5 mice per group. (E) Flow cytometry of IL-12+ cells as a proportion of CD45+ cells, using IL-12p40 reporter mice. (F) MC38 tumor bearing IL-12p40 reporter mice were treated with aPD-1, with or without co-administration of aIFN-!. Tumors were collected for flow cytometry and DC populations were defined as CD45+F4/80−CD11chi MHCIIhi. Shown are two representative plots of control and aIFN-! conditions from n=5 per group, data correspond to FIG. 3D. (G) Tumor growth of indicated animals at 3 days post aPD-1 treatment with or without aIFN-!. Tumor size of each individual animal defines pre-treatment baseline and values reported are changes from baseline after treatment; n=5 mice per group. *p-value<0.05, ****p<0.001 Student's two-tailed, t-test.

FIGS. 11A-E. IL-12 Responses to aPD-1 mAbs Do Not Occur in the Lymph Node and aPD-1 Treatment Functions Independently of Lymphocyte Recirculation. (A) MC38 tumor-bearing IL-12 reporter mice were treated with aPD-1 or not (control), and tumor-draining lymph nodes were harvested 48 hours after treatment. Flow cytometry of IL-12+ DCs is shown with control (grey) and aPD-1 (black) treatments; n=4 mice/group. (B) MC38 tumor-bearing IFN-! reporter mice were treated with aPD-1 or not (control) and tumor-draining lymph nodes were harvested 48 hours after treatment. Flow cytometry of IFN-!+ cells is shown with control (grey) and aPD-1 (black) treatments; n≥3 mice/group. (C) Single cell RNA sequencing expression data of the proliferation associated genes Rrm2 and Mki67 within tumor immune cell populations. Comparisons are from samples treated or not with aPD-1. Cell clusters positive for either Rrm2 or Mki67 are also shown to express Cd8a. (D) Blood of aPD-1-treated animals without (black) or with FTY720 was analyzed by flow cytometry for circulating CD8+ T cells; n≥7 mice/group. (E) Tumor growth curves of MC38 tumor-bearing mice that received FTY720 alone (grey circle), aPD-1 alone (black square), both aPD-1 and FTY720 (grey square), or that were left untreated (control, grey circle); n≥6 mice/group from one experiment. n.s=not significant, ***p<0.001. One way ANOVA with multiple comparisons.

FIG. 12. Flow Cytometry Sorting Strategy and Validation of Human Tumor Infiltrating Lymphocytes. Fresh tumor samples isolated from cancer patients were mechanically dissociated and digested into single cell suspensions, and the representative flow cytometry gating strategies for isolating CD8+ T cells. Samples were re-run through the initial gating strategy to ensure sample purity.

FIGS. 13A-C. IL-12 Expressing Cells Express More CD40 and AZD5882 can Induce IL-12 Production In vitro. (A) Flow cytometry of MC38 tumors from IL-12p40-eYFP reporter mice, stained for CD40 expression; n=7 per group. (B) Flow cytometry of CD40 expression from the following tumor immune cell populations: Non-Antigen Presenting Cells (non-APCs, defined as F4/80−CD11c−MHCII−), macrophages (F4/80+) and IL-12+ DCs (CD11chi MHCIIhi IL-12+); n=4 per group. (C) Flt3L-derived bone marrow DCs were cultured in vitro with various concentrations of AZD5582 for 24 hours, and were harvested for RNA. Shown is fold change expression of IL-12p40 transcripts compared to untreated conditions (n=3 per condition). Results are representative of at least 2 independent experiments. ****p<0.0001; Student's two tailed t-test.

FIGS. 14A-G MC38 and B16 F10 Tumor Response to aPD-1+aCD40 Combination Therapy. (A) Bone marrow chimeras reconstituted with either NIK KO or WT bone marrow were implanted with MC38 tumors and treated with aPD-1. NIK KO reconstituted mice not treated with aPD-1 were used as additional controls. The plot shown below indicates tumor progression over time in the different experimental groups (n=5-10 mice/group). (B, C) MC38 tumor growth in mice that received aPD-1 mAb, agonistic aCD40 mAb or aPD-1+aCD40 combination. Untreated mice were used as controls. Tumors were approximately 75 mm3 in size at initiation of treatment (n≥6 mice/group). (B) shows tumor volumes; dots for each group represent single mice. (C) shows percent change tumor volume when compared to pre-treatment data. (D) MC38 bearing animals that showed a complete response to aPD-1+aCD40 combination treatment were re-challenged with MC38 tumor cell implantation 50 days following initial tumor rejection. Naive mice that had not been exposed to MC38 were used as controls (n=7 mice/group). Data show the percentage of mice rejecting MC38 re-challenge. (E and F) B16F10 tumor growth in mice that received aPD-1 mAb, agonistic aCD40 mAb or aPD-1+aCD40 combination. Untreated mice were used as controls. Tumors were approximately 75 mm3 in size at initiation of treatment (n≥6 mice/group). (E) shows tumor volumes; dots for each group represent single mice. (F) shows percent change tumor volume when compared to pre-treatment data. (G) B16F10 tumor volume measurements in mice that received aCD40, aPD-1+aCD40 or aPD-1+aCD40+aIL-12. Untreated mice served as controls. Dots for some groups represent single mice. n≥5 mice/group. Results are representative of at least 2 independent experiments. *p<0.05, **p<0.01, ***p<0.001, One way ANOVA with multiple comparisons.

### DETAILED DESCRIPTION

Selective inability to activate non-canonical NFkB in hematopoietic cells renders animal models unresponsive to cancer immunotherapy. Activating non-canonical NFkB through drugs not previously associated to non-canonical NFkB activation in dendritic cells can elicit anti-tumor immune responses.

To date, FDA-approved therapeutics targeting the PD-1-PDL1 signaling axis, in particular aPD-1 mAbs, have proved efficacious in the clinic among immune checkpoint blockade therapies. The ability of these drugs to drive sustained tumor control depends on several variables, including tumor infiltration by CD8+ T cells (Galon et al., 2013; Huang et al., 2017), interferon γ (IFN-γ) production (Schreiber et al., 2011; Ayers et al., 2017), neoantigen abundance (Rizvi et al., 2015), MHC class I expression (Marty et al., 2017; McGranahan et al., 2017), CD28 co-stimulatory signals (Hui et al., 2017; Kamphorst et al., 2017), patient microbiota (Matson et al., 2018; Routy et al., 2018) and antibody composition (Arlauckas et al., 2017; Dahan et al., 2015). However, we still have a limited understanding of how immune checkpoint blockers engage complex tumor microenvironments and which mechanisms define treatment success during the time when tumor rejection occurs.

To address these knowledge gaps, we sought to track key readouts of immunotherapy function in vivo at single cell resolution (Pittet et al., 2018) and during tumor rejection, and decipher how immune-mediated tumor control is achieved. Considering that IFN-γ and interleukin 12 (IL-12) are key immune players in tissue-specific destruction (Galon et al., 2013; Nastala et al., 1994), we used intravital imaging to track these factors within tumors following aPD-1 treatment. Complementing single cell imaging, we also used single cell RNA sequencing (scRNAseq) to provide an unbiased view of immunotherapeutic responses across the tumor immune microenvironment.

These approaches, further combined with manipulations of the IFN-γ and IL-12 pathways in vivo, indicated that aPD-1 drove IL-12 production by a subset of tumor-infiltrating dendritic cells (DCs). Our imaging platform identified that DC activation was indirect (the drug did not detectably bind these cells in vivo) but required DC sensing of IFN-γ, which was produced by aPD-1-activated T cells. In turn, IL-12 produced by DCs licensed effector T cell responses. We further report that the non-canonical nuclear factor kappa-light-chain-enhancer of activated B cells (NFkB) pathway was enriched within IL-12-producing DCs. This pathway was required for response to aPD-1, and agonizing it in a therapeutic setting enhanced IL-12 production by tumor-infiltrating DCs.

We used single cell resolution readouts, including intravital microscopy and scRNAseq, to discover cancer immunotherapy pharmacodynamics within tumors and better define in vivo mechanisms of tumor rejection. We found that the antitumor cytokines IFN-γ and IL-12 were mutually induced by immunotherapy and further distinguished direct and indirect mechanisms of activation for these respective cytokines. Principally, we identified that aPD-1 directly induced IFN-γ production by activated T cells, but indirectly induced IL-12 production by a subset of intratumoral DCs. IL-12 production required DC sensing of IFN-γ, and, in turn, licensed effector T cell responses in both mice and cancer patients. We also showed that IL-12-producing DCs were enriched for non-canonical NFkB signaling pathway components, that the critical non-canonical NFkB kinase NIK was required for aPD-1 response, and that agonism of the non-canonical NFkB pathway in a therapeutic setting produced an IL-12-dependent antitumor response. Furthermore, triggering the T cell:DC crosstalk through non-canonical NFkB agonism in combination with aPD1 treatment could potently enhance antitumor immunity. These data support an IL-12-driven “licensing” model of aPD-1 therapy, in which aPD-1 mAb targeting of T cells leads to tumor elimination only after successful crosstalk between these T cells and DCs. We suggest further that responses to immunotherapy can be improved through rational drug combinations that accentuate the crosstalk between lymphoid and myeloid immune compartments.

Real-time in vivo imaging allows one to identify not only primary targets of immunotherapeutics (drug pharmacokinetics) (Arlauckas et al., 2017) but also how the tumor microenvironment responds to treatment (drug pharmacodynamics). Consequently, this type of imaging complements the use of gene-deficient mouse models to study cancer treatments: whereas gene-deficient models can establish the relevance of particular genes in immunotherapy, imaging provides molecular dynamics at single cell and spatial resolutions and over a longitudinal course of therapeutic response. Caveats still exist with this imaging approach however as distribution and effector functions of antibodies may differ between species and antibody compositions. It is also worth noting that the investigations presented in this study used cytokine reporter animals for readout of immune cells' functional attributes, as opposed to immune cells' identities. We believe this is important because antitumor immune functions may not necessarily be cell type-dependent, so in theory different cell types can be imaged but the functional readout still remains. For example, in the experimental setups used in this study we found that CD8⁺ T cells and DCs are the primary producers of IFN-γ and IL-12, respectively; however, under different experimental contexts it is possible that NK cells may also produce IFN-γ and macrophages may also produce IL-12. It should also be noted that the present report focuses on pharmacodynamic imaging of aPD-1 and aCD40, although our imaging platform can in principle be used to interrogate any immune drug or other therapeutic agent, and further be expanded to additional functional readouts.

There is increasing support for DCs taking a center stage in checkpoint immunotherapies in cancer. In particular, the cDC1 subtype of DCs, which resembles the DC1 subtype presented here, is adept at cross-presenting antigens (Schlitzer and Ginhoux, 2014) and appears essential for T cell-driven antitumor immunity (Hildner et al., 2008) Interestingly, these DCs may be involved at different stages during the tumor rejection process: besides their critical role for priming T cells in lymph nodes (Martin-Fontecha et al., 2003), recent studies demonstrated that DCs can be found in tumors, where they recruit T cells and stimulate tumor-reactive T cell responses locally (Spranger et al., 2014; de Mingo Pulido et al., 2018). The findings presented here align with the notion that intratumoral DCs can exhibit key antitumor functions and promote aPD-1 immunotherapy. Systemic involvement of immunotherapy responses could also be relevant. For example, in the context of a longer duration of response, it is possible that aPD-1's antitumor activity is promoted initially by intratumoral DCs and T cells, and later by an additional pool of cells that are recruited from outside the tumor microenvironment (perhaps from the bone marrow or even from tumor-draining lymph nodes).

We found that IL-12⁺ DCs do not always express the marker CD103 (encoded by Itgae), which is often used to define antitumor DCs. It is possible that CD103 is not required for DCs' antitumor functions and that its expression depends at least in part on the tissue where the DCs reside. In contrast, IL-12 may be both a marker and functional feature of immunostimulatory tumor DCs, based on our findings that i) IL-12⁺ DCs share many features with cross-presenting DC1 cells, including expression of Batf3, Irf8, Flt3, and Ly75 (DEC205), and ii) IL-12 is required for immunotherapy efficacy. This notion further accords with prior evidence that cross-presenting tumor DCs have elevated IL-12 expression (Broz et al., 2014; Ruffell et al., 2014). Our data further indicate that IL-12-producing DCs can be generated by circulating precursors, although future studies should aim to precisely determine the ontogeny of these cells.

The findings presented here show that IL-12 cytokine signals supplied by intratumoral DCs assist antitumor immunity. It will be interesting to further investigate the interactions between IL-12⁺ DCs, IFN-γ⁺ T cells and immunotherapeutics. For example, considering that DCs can express PD-L1 and that PD-1 is activated upon binding to PDL1, it should be helpful to elucidate the function and fate of PDL1 expressed by intratumoral DCs following aPD(L)1 treatment. Also, since IL-12⁺ DCs express the highest levels of CD28's co-stimulatory ligands, CD80 and CD86, it is possible that these ligands contribute to an aPD-(L)1 antitumor response. Furthermore, IL-12 produced by intratumoral DCs may mediate antitumor effects through regulation of transcription factors such as T-bet and Eomes in effector T cells. Indeed, IL-12 may activate T-bet (Joshi et al., 2007; Szabo et al., 2000) and in doing so subvert exhaustion phenotypes (Kao et al., 2011). IL-12 may also repress Eomes (Takemoto et al., 2006), which is a major regulator of T cell exhaustion (Paley et al., 2012). Further study of cells responding to IL-12 could define additional avenues to reverse T cell exhaustion and potentiate antitumor immunity.

By looking at direct versus indirect effects of immunotherapy in the tumor microenvironment we can start to better understand the mechanisms of tumor rejection in vivo, and, by extension, to rationally design combination therapeutic strategies. Here, we initially used the MC38 mouse tumor model because it is sensitive to aPD-1 treatment and thus is relevant to define mechanisms dictating treatment success. Furthermore, recapitulation of IFN-γ/IL-12 positive feedback mechanisms, through combination therapy, enables tumor control in harder-to-treat cancer models. Specifically, our analysis demonstrated that activating the non-canonical NFkB pathway in intratumoral DCs through either CD40 agonism or cIAP inhibition, can potently enhance aPD-1-mediated tumor control.

Treatments combining CD40 agonists with PD-1 pathway inhibitors (NCT03123783, NCT02376699), and cIAP inhibitors with aPD-L1 (NCT03270176), are currently in clinical trials. We suggest that both treatment strategies may rely upon the non-canonical NFkB pathway and DCs. Further, since our studies indicated that non-canonical NFkB-targeting drugs depend upon IL-12 for mediating antitumor activity, we speculate that introduction of IL-12 could potently enhance aPD-1 immunotherapy. Previous attempts to develop IL-12-based therapies for human use had severely toxic consequences (Lasek et al., 2014) likely due to systemic administration routes. However, targeted intratumoral delivery of IL-12 encoding plasmids is safe and has already demonstrated antitumor efficacy as monotherapy (Daud et al., 2008). We suggest that further clinical studies should test whether rationally designed therapeutic strategies that accentuate T cell:DC crosstalk can enforce tumor-eliminating positive feedback mechanisms and expand the proportion of cancers sensitive to immunotherapy.

### Methods of Treatment

In view of the discovery that inhibiting the non-canonical NFkB pathway, e.g., by inhibiting the kinase NIK in combination with immunotherapy greatly enhances antitumor immunity, provided herein are methods of treating a cancer in a subject that include administering an inhibitor of the non-canonical NFkB pathway, e.g., of NIK, in combination with an immunotherapy. Specific embodiments and various aspects of these methods are described below.

Methods of Treating Cancer

The methods generally include identifying a subject who has a tumor, e.g., a cancer. As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer will be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. While the present study focused on pancreatic cancer because of its dismal prognosis and the lack of progress against its metastatic form, the present compositions and methods are broadly applicable to solid malignancies. Thus the cancer can be of any type of solid tumor, including but not limited to: breast, colon, kidney, lung, skin, ovarian, pancreatic, rectal, stomach, thyroid, or uterine cancer.

Tumors include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, cancers evaluated or treated by the methods described herein include epithelial cancers, such as a lung cancer (e.g., non-small-cell lung cancer (NSCLC)), breast cancer, colorectal cancer, kidney cancer, head and neck cancer, prostate cancer, pancreatic cancer (e.g., Pancreatic ductal adenocarcinoma (PDAC)) or ovarian cancer. Epithelial malignancies are cancers that affect epithelial tissues.

A cancer can be diagnosed in a subject by a health care professional (e.g., a physician, a physician's assistant, a nurse, or a laboratory technician) using methods known in the art. For example, a metastatic cancer can be diagnosed in a subject, in part, by the observation or detection of at least one symptom of a cancer in a subject as known in the art. A cancer can also be diagnosed in a subject using a variety of imaging techniques (e.g., alone or in combination with the observance of one or more symptoms of a cancer in a subject). For example, the presence of a cancer can be detected in a subject using computer tomography, magnetic resonance imaging, positron emission tomography, and X-ray. A cancer can also be diagnosed by performing a biopsy of tissue from the subject. A cancer can also be diagnosed from serum biomarkers, such as CA19.9, CEA, PSA, etc.

In some embodiments, the methods can include determining whether the cancer expresses or overexpresses an immune checkpoint molecule, e.g., PD-L1. Methods for detecting expression of an immune checkpoint molecule, e.g., PD-L1 in a cancer, e.g., in a biopsy or other sample comprising cells from the cancer, are known in the art, e.g., including commercially available or laboratory-developed immunohistochemistry (IHC); see, e.g., Udall et al., Diagn Pathol. 2018; 13: 12. The level can be compared to a threshold or reference level, and if a level of expression of an immune checkpoint molecule, e.g., PD-L1 above the threshold or reference level are seen, the subject can be selected for a treatment as descried herein. In some embodiments, the methods can include determining whether the cancer has high levels of microsatellite instability (MSI), e.g., as described in Kawakami et al., Curr Treat Options Oncol. 2015 July; 16(7):30; Zeinalian et al., Adv Biomed Res. 2018; 7: 28, and selecting for treatment a cancer that is MSI-high or that has levels of MSI above a threshold or reference level.

A treatment comprising any one or more of the inhibitor of the non-canonical NFkB pathway, e.g., of NIK, as described herein, optionally in combination with an immunotherapy, as described herein, can be administered to a subject having cancer. The treatment can be administered to a subject in a health care facility (e.g., in a hospital or a clinic) or in an assisted care facility. In some embodiments, the subject has been previously diagnosed as having a cancer. In some embodiments, the subject has already received therapeutic treatment for the cancer. In some embodiments, one or more tumors has been surgically removed prior to treatment as described herein.

In some embodiments, the administering of at least one inhibitor of the non-canonical NFkB pathway, e.g., of NIK, as described herein, in combination with an immunotherapy, results in a decrease (e.g., a significant or observable decrease) in the size of a tumor, a stabilization of the size (e.g., no significant or observable change in size) of a tumor, or a decrease (e.g., a detectable or observable decrease) in the rate of the growth of a tumor present in a subject. A health care professional can monitor the size and/or changes in the size of a tumor in a subject using a variety of different imaging techniques, including but not limited to: computer tomography, magnetic resonance imaging, positron emission tomography, and X-ray. For example, the size of a tumor of a subject can be determined before and after therapy in order to determine whether there has been a decrease or stabilization in the size of the tumor in the subject in response to therapy. The rate of growth of a tumor can be compared to the rate of growth of a tumor in another subject or population of subjects not receiving treatment or receiving a different treatment. A decrease in the rate of growth of a tumor can also be determined by comparing the rate of growth of a tumor both prior to and following a therapeutic treatment (e.g., treatment with any of the inhibitors of the non-canonical NFkB pathway, e.g., of NIK, as described herein, in combination with an immunotherapy, as described herein). In some embodiments, the visualization of a tumor can be performed using imaging techniques that utilize a labeled probe or molecule that binds specifically to the cancer cells in the tumor (e.g., a labeled antibody that selectively binds to an epitope present on the surface of the cancer cell).

In some embodiments, administering an inhibitor of the non-canonical NFkB pathway, e.g., of NIK, in combination with an immunotherapy, to the subject decreases the risk of developing a metastatic cancer (e.g., a metastatic cancer in a lymph node) in a subject having (e.g., diagnosed as having) a primary cancer (e.g., a primary breast cancer) (e.g., as compared to the rate of developing a metastatic cancer in a subject having a similar primary cancer but not receiving treatment or receiving an alternative treatment). A decrease in the risk of developing a metastatic tumor in a subject having a primary cancer can also be compared to the rate of metastatic cancer formation in a population of subjects receiving no therapy or an alternative form of cancer therapy.

A health care professional can also assess the effectiveness of therapeutic treatment of a cancer by observing a decrease in the number of symptoms of cancer in the subject or by observing a decrease in the severity, frequency, and/or duration of one or more symptoms of a cancer in a subject. A variety of symptoms of a cancer are known in the art and are described herein.

In some embodiments, the administering can result in an increase (e.g., a significant increase) in lifespan or chance of survival or of a cancer in a subject (e.g., as compared to a population of subjects having a similar cancer but receiving a different therapeutic treatment or no therapeutic treatment). In some embodiments, the administering can result in an improved prognosis for a subject having a cancer (e.g., as compared to a population of subjects having a similar cancer r but receiving a different therapeutic treatment or no therapeutic treatment).

Immunotherapy

The methods can also include administering an immunotherapy, e.g., an immune checkpoint inhibitor; cancer vaccines; dendritic cell vaccine; adaptive T cell therapy; and/or chimeric antigen receptor-expressing immune effector cells, e.g., CAR-T cells. In preferred embodiments, the immunotherapy results in an increase in IFNγ activity and/or levels.

Currently approved immune checkpoint inhibitors include monoclonal antibodies (mAbs) that target the programmed cell death protein 1 (PD-1)/PD-L1/2 or cytotoxic T-lymphocyte-associated protein 4 (CTLA-4) pathways, and agents targeting other pathways are in clinical development (including OX40, Tim-3, and LAG-3) (See, e.g., Leach et al., Science 271, 1734-1736 (1996); Pardoll, Nat. Rev. Cancer 12, 252-264 (2012); Topalian et al., Cancer Cell 27, 450-461 (2015); Mahoney et al., Nat Rev Drug Discov 14, 561-584 (2015)). The present methods can include the administration of checkpoint inhibitors such as antibodies including anti-CD137 (BMS-663513); anti-PD-1 (programmed cell death 1) antibodies (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538)); anti-PDL1 (programmed cell death ligand 1) or anti-PDL2 (e.g., BMS-936559, MPDL3280A, atezolizumab, avelumab and durvalumab); or anti-CTLA-4 (e.g., ipilumimab or tremelimumab). See, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells:

Alternatively or in addition, the immunotherapy can include administration of a population of immune effector cells (e.g., T cells or Natural Killer (NK) cells) that can be engineered to express one or more Chimeric Antigen Receptors (CARs). CARs are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621). Such T cells are referred to as CAR-T cells. See, e.g., US20180355052A1; WO2017117112A1; US20190309307; US20190292246; US 20190298772; US20190000880; US20150376296; Bollino and Webb, “Chimeric antigen receptor-engineered natural killer and natural killer T cells for cancer.” Immunotherapy. Translational Research 2017; 187:32-43; Fu and Tang, Recent Patents on Anti-Cancer Drug Discovery, 14(1), 2019; 14(1):60-69, DOI: 10.2174/1574892814666190111120908; Jürgens and Clarke, Nature Biotechnology 37:370-375 (2019); and references cited therein. In some embodiments, the CAR-T cells are autologous, i.e., derived from the same individual to whom it is later to be re-introduced e.g., during therapy. In some embodiments, the CAR-T cells are non-autologous, i.e., derived from a different individual relative to the individual to whom the material is to be introduced. Alternatively, a T cell-engaging therapeutic agent, such as a bispecific or other multispecific agent, e.g., antibody that is capable of recruiting and/or engaging the activity of one or more T cells, such as in a target-specific manner, can be used (see US 20190292246).

A number of immunotherapies are known in the art. In some embodiments, these therapies may primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethylene ATP trisodium salt), 8-(4-chlorophenylthio) cAMP (pCPT-cAMP) and a related cyclic nucleotide analog (8-[4-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013). In another example, M2 macrophage targeted therapy includes clodronate-liposomes (Zeisberger, et al., Br J Cancer. 95:272-281 (2006)), DNA based vaccines (Luo, et al., J Clin Invest. 116(8): 2132-2141 (2006)), and M2 macrophage targeted pro-apoptotic peptides (Cieslewicz, et al., PNAS. 110(40): 15919-15924 (2013)). Immnotherapies that target Natural Killer T (NKT) cells can also be used, e.g., that support type I NKT over type II NKT (e.g., CD1d type I agonist ligands) or that inhibit the immune-suppressive functions of NKT, e.g., that antagonize TGF-beta or neutralize CD1d.

Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4:734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g. fresolimumab, Infliximab, Lerdelimumab, GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPS” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin. See, Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-AB (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3:e28811). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7. In some embodiments, the methods include administering a composition comprising tumor-pulsed dendritic cells, e.g., as described in WO2009/114547 and references cited therein. See also Shiao et al., Genes & Dev. 2011. 25: 2559-2572.

For further information regarding immunotherapies that can be used in the present methods, see Christofi et al., Cancers (Basel). 2019 Sep. 30; 11(10). pii: E1472; Demaria et al., Nature. 2019 October; 574(7776):45-56; and Bastien et al., Seminars in Immunology 42 (2019) 101306, and references cited therein.

Inhibitors of the Non-Canonical NFkB Pathway

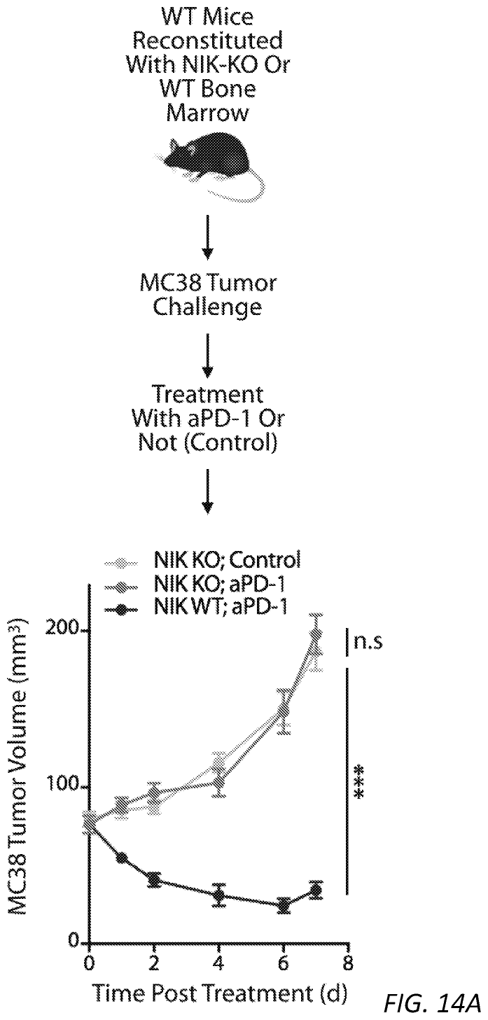

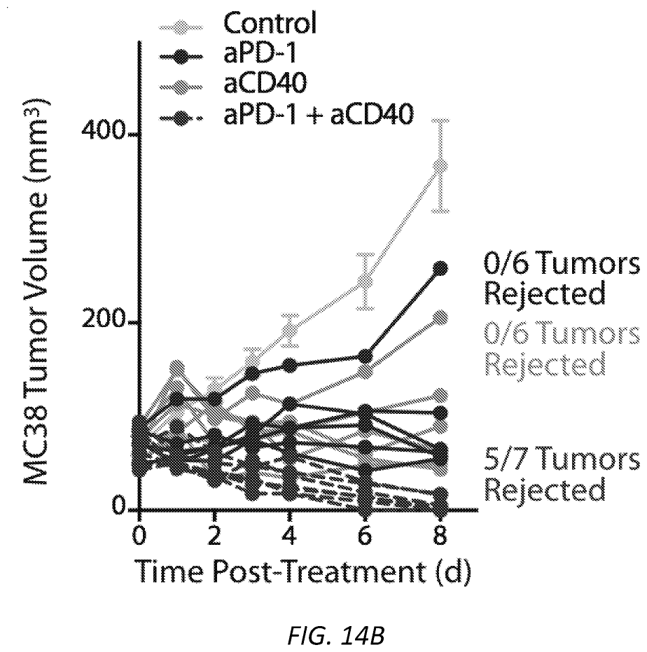

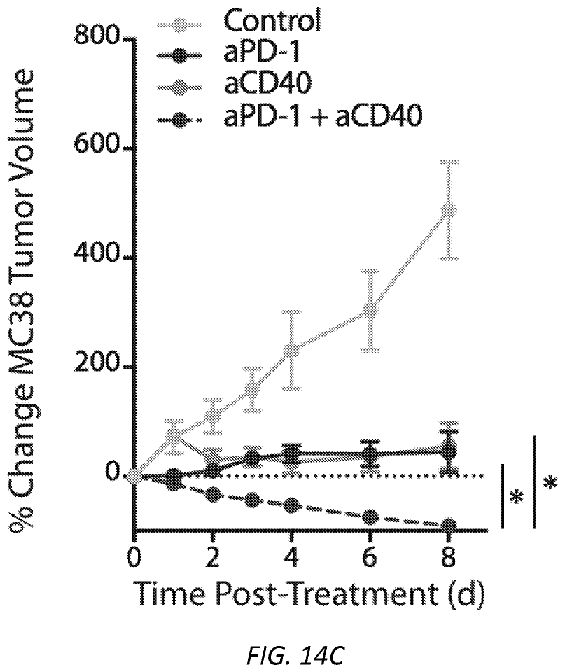

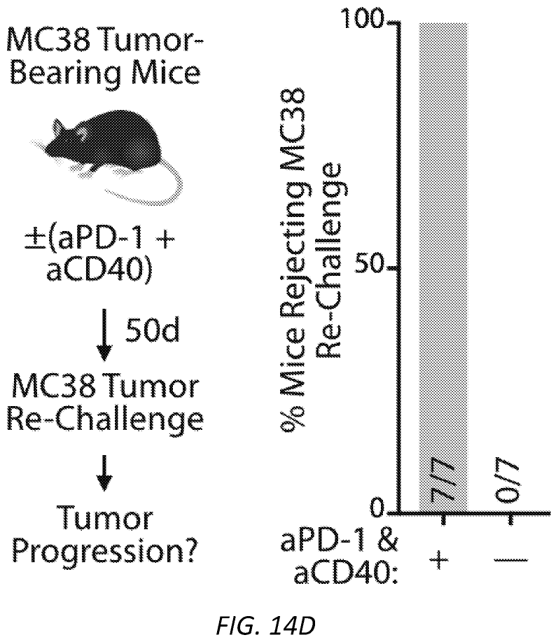

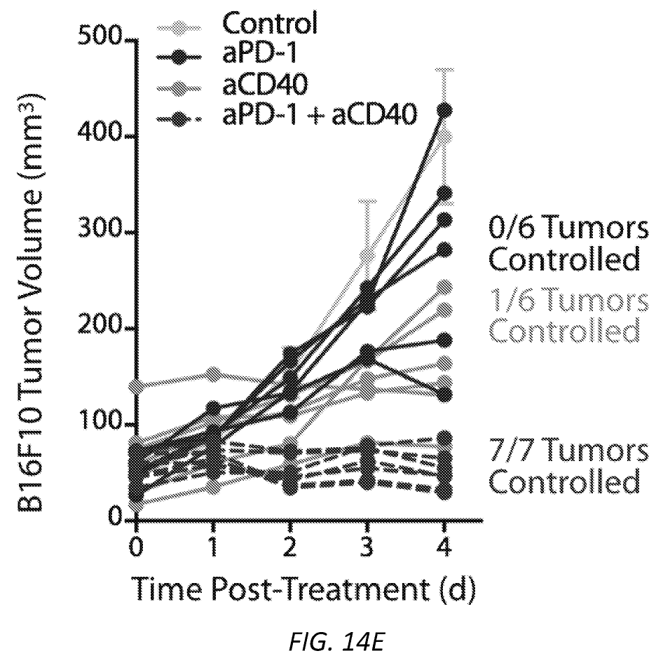

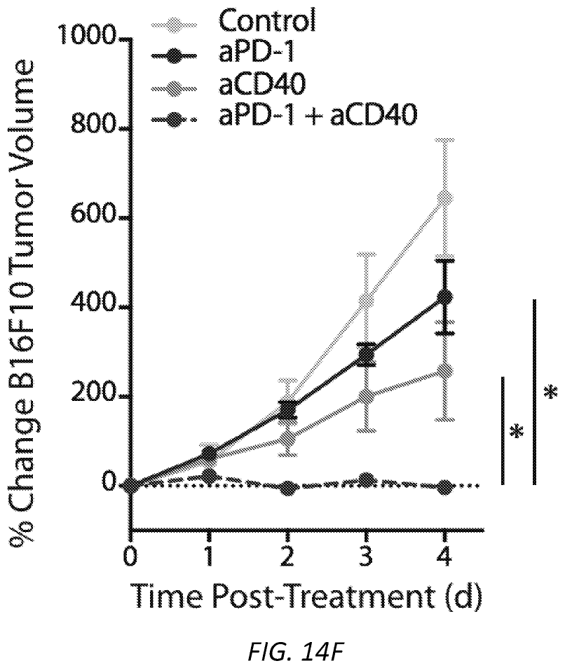

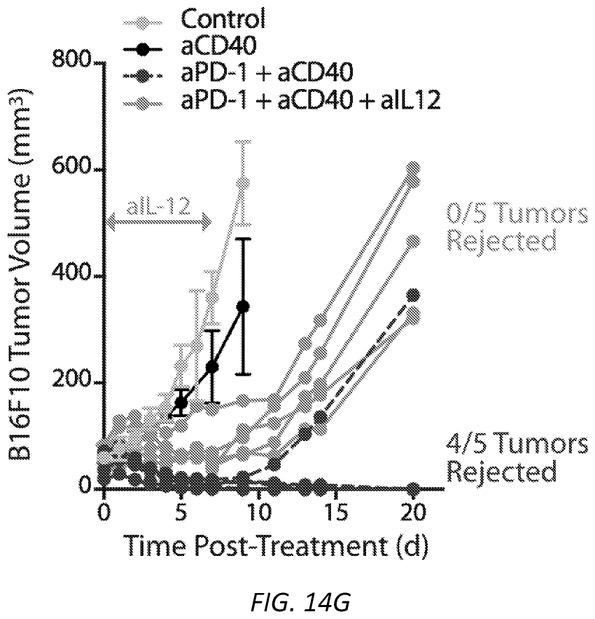

In some embodiments, the inhibitor of the non-canonical NFkB pathway is a NIK inhibitor. NIK inhibitors include, but are not limited to, alkynyl alcohols (as disclosed in WO2009158011); 6-membered heteroaromatic substituted cyanoindoline derivatives (as disclosed in WO2017125534); pyrazoloisoquinoline derivatives (as disclosed in JP2017031146); the compounds disclosed in FIG. 14 of WO2013014244; 6-azaindole aminopyrimidine derivatives (as disclosed in US20110183975); a polypeptide that blocks NIK-HC8 binding (as disclosed in U.S. Pat. No. 8,338,567); pyrazoloisoquinoline derivatives (as disclosed in U.S. Pat. No. 6,841,556); candidate inhibitors listed in Table 1 of Wang et al., including sulfapyridine and propranolol (as disclosed in Wang et al. (2018). Sci Report, 8: 1657); tricyclic NF-κB inducing kinase inhibitors (as disclosed in Castanedo et el. (2017). J Med Chem, 60 (3): 627-640, e.g., compound 10 (9-fluoro-10-[(3R)-3-hydroxy-3-(5-methylisoxazol-3-yl)but-1-ynyl]-5,6-dihydroimidazo[1,2-d][1,4]benzoxazepine-2-carboxamide), 32 (10-fluoro-9-[(3R)-3-hydroxy-3-(5-methyl-1,2-oxazol-3-yl)but-1-yn-1-yl]-2,5-diazatetracyclo[11.1.1.02,6.07,12]pentadeca-3,5,7(12),8,10-pentaene-4-carboxamide), or 33 (10-[(3R)-3-hydroxy-3-(5-methylisoxazol-3-yl)but-1-ynyl]-N3-methyl-5,6-dihydroimidazo[1,2-d][1,4]benzoxazepine-2,3-dicarboxamide); 4H-isoquinoline-1,3-dione and 2,7-naphthydrine-1,3,6,8-tetrone (as disclosed in Mortier et al. (Mortier et al. (2010). Bioorganic & Medicinal Chemistry Letters, 20 (15): 4515-4520)); and/or N-Acetyl-3-aminopyrazoles (as disclosed in Pippione et al. (2018). Medchemcomm. 9(6): 963-968). NIK inhibitors available from commercial suppliers, include, but are not limited to NIK-SMI1 ((R)-6-(3-((3-hydroxy-1-methyl-2-oxopyrrolidin-3-yl)ethynyl)phenyl)-4-methoxypicolinamide, Cat. No. PC-62514, ProbeChem), AM-0216 ((R)-4-(1-(2-aminopyrimidin-4-yl)indolin-6-yl)-2-(thiazol-2-yl)but-3-yn-2-ol, Cat. No. PC-35550. ProbeChem), AM-0561 ((R)-4-(3-(2-amino-5-chloropyrimidin-4-yl)imidazo[1,2-a]pyridin-6-yl)-2-(thiazol-2-yl)but-3-yn-2-ol, Cat. No. PC-35549, ProbeChem), or Amgen16 (1-((1-(2-amino-5-chloropyrimidin-4-yl)indolin-6-yl)ethynyl)cyclopentan-1-ol, Cat. No. PC-35548, ProbeChem).

Methods of identifying additional NIK inhibitors are known in the art, see e.g., US20140234870; WO2013014244; Mortier et al., 2010 (supra); Hassan, N. J. et al. (Biochem J 2009, 419:65-73); Wang et al., 2018 (supra).

Dosing, Administration, and Compositions

In any of the methods described herein, the inhibitor of the non-canonical NFkB pathway, e.g., of NIK, in combination with an immunotherapy, can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy.

In some embodiments of any of the methods described herein, inhibitor of the non-canonical NFkB pathway, e.g., of NIK, in combination with an immunotherapy, is administered to a subject that has been diagnosed as having a cancer. In some embodiments, the subject has been diagnosed with melanoma; brain cancer, e.g., GBM; breast cancer; or pancreatic cancer. In some non-limiting embodiments, the subject is a man or a woman, an adult, an adolescent, or a child. The subject can have experienced one or more symptoms of a cancer or metastatic cancer (e.g., a metastatic cancer in a lymph node). The subject can also be diagnosed as having a severe or an advanced stage of cancer (e.g., a primary or metastatic cancer). In some embodiments, the subject may have been identified as having a metastatic tumor present in at least one lymph node. In some embodiments, the subject may have already undergone surgical resection, e.g., partial or total pancreatectomy, lymphectomy and/or mastectomy.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the inhibitors of the non-canonical NFkB pathway, e.g., of NIK, as described herein, optionally in combination with an immunotherapy, or pharmaceutical compositions described herein. In any of the methods described herein, the at least one inhibitory nucleic acids or pharmaceutical composition (e.g., any of the inhibitory nucleic acids or pharmaceutical compositions described herein) can be administered intravenously, intraarterially, subcutaneously, intraperitoneally, or intramuscularly to the subject. In some embodiments, the at least one inhibitory nucleic acids or pharmaceutical composition is directly administered (injected) into or adjacent to (e.g., within 6″, 5″, 4″, 3″ 2″, or 1″ of) a tumor or lymph node in a subject.

In some embodiments, the subject is administered at least one inhibitor of the non-canonical NFkB pathway, e.g., of NIK, as described herein, in combination with an immunotherapy, or pharmaceutical composition (e.g., any of the inhibitors of the non-canonical NFkB pathway, e.g., of NIK, as described herein, in combination with an immunotherapy or pharmaceutical compositions described herein) and at least one additional therapeutic agent. The at least one additional therapeutic agent can be a chemotherapeutic agent. By the term “chemotherapeutic agent” is meant a molecule that can be used to reduce the rate of cancer cell growth or to induce or mediate the death (e.g., necrosis or apoptosis) of cancer cells in a subject (e.g., a human). In non-limiting examples, a chemotherapeutic agent can be a small molecule, a protein (e.g., an antibody, an antigen-binding fragment of an antibody, or a derivative or conjugate thereof), a nucleic acid, or any combination thereof. Non-limiting examples of chemotherapeutic agents include one or more alkylating agents; anthracyclines; cytoskeletal disruptors (taxanes); epothilones; histone deacetylase inhibitors; inhibitors of topoisomerase I; inhibitors of topoisomerase II; kinase inhibitors; nucleotide analogs and precursor analogs; peptide antibiotics; platinum-based agents; retinoids; and/or vinca alkaloids and derivatives; or any combination thereof. In some embodiments, the chemotherapeutic agent is a nucleotide analog or precursor analog, e.g., azacitidine; azathioprine; capecitabine; cytarabine; doxifluridine; fluorouracil; gemcitabine; hydroxyurea; mercaptopurine; methotrexate; or tioguanine. Other examples include cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

In some embodiments, the chemotherapeutic agent is chosen based on the cancer type or based on genetic analysis of the cancer; for example, for pancreatic cancer, one or more of ABRAXANE (albumin-bound paclitaxel), Gemzar (gemcitabine), capecitabine, 5-FU (fluorouracil) and ONIVYDE (irinotecan liposome injection), or combinations thereof, e.g., FOLFIRINOX, a combination of three chemotherapy drugs (5-FU/leucovorin, irinotecan and oxaliplatin), or modified FOLFIRINOX (mFOLFIRINOX) can be administered. Further combinations of targets that may work synergistically by complementary mechanisms could be used. For example, combination therapies can be used that physically alter the tumor microenviroment by enzymatic degradation via recombinant human hyaluronidase (PEGPH20),^{30,31} or other alternative chemotherapy agents, and/or alternative checkpoint inhibitors that may promote a synergistic effect in activating T-cells (e.g., anti-CTLA-4).

The methods and compositions can also include administration of an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, at least one additional therapeutic agent and at least one inhibitors of the non-canonical NFkB pathway, e.g., of NIK, as described herein, in combination with an immunotherapy, are administered in the same composition (e.g., the same pharmaceutical composition). In some embodiments, the at least one additional therapeutic agent and the at least one inhibitor of the non-canonical NFkB pathway, e.g., of NIK, in combination with an immunotherapy, are administered to the subject using different routes of administration (e.g., at least one additional therapeutic agent delivered by oral administration and at least one inhibitor of the non-canonical NFkB pathway, e.g., of NIK, as described herein, in combination with an immunotherapy, delivered by intravenous administration).

In any of the methods described herein, the at least one inhibitor of the non-canonical NFkB pathway, e.g., of NIK, as described herein, optionally in combination with an immunotherapy, and, optionally, at least one additional therapeutic agent can be administered to the subject at least once a week (e.g., once a week, twice a week, three times a week, four times a week, once a day, twice a day, or three times a day). In some embodiments, at least two different inhibitors of the non-canonical NFkB pathway, e.g., of NIK, in combination with an immunotherapy, are administered in the same composition (e.g., a liquid composition). In some embodiments, at least one inhibitor of the non-canonical NFkB pathway, e.g., of NIK, in combination with an immunotherapy, and at least one additional therapeutic agent are administered in the same composition (e.g., a liquid composition). In some embodiments, the at least one inhibitors of the non-canonical NFkB pathway, e.g., of NIK, in combination with an immunotherapy, and the at least one additional therapeutic agent are administered in two, three or more different compositions (e.g., a first, e.g., liquid, composition containing at least one inhibitor of the non-canonical NFkB pathway, e.g., of NIK, as described herein, in combination with or separate from the composition comprising the immunotherapy, and a second or third, e.g., solid oral, composition containing at least one additional therapeutic agent). In some embodiments, the at least one additional therapeutic agent is administered as a pill, tablet, or capsule. In some embodiments, the at least one additional therapeutic agent is administered in a sustained-release oral formulation. In some embodiments, any one or more of the agents, e.g., the inhibitor of the non-canonical NFkB pathway, the immunotherapy, or the at least one additional therapeutic agent, is administered as an injection or intravenous infusion.

In some embodiments, the one or more additional therapeutic agents can be administered to the subject prior to administering the at least one inhibitors of the non-canonical NFkB pathway, e.g., of NIK, as described herein, optionally in combination with an immunotherapy. In some embodiments, the one or more additional therapeutic agents can be administered to the subject after administering the at least one inhibitors of the non-canonical NFkB pathway, e.g., of NIK, as described herein, optionally in combination with an immunotherapy. In some embodiments, the one or more additional therapeutic agents and the at least one inhibitors of the non-canonical NFkB pathway, e.g., of NIK, as described herein, optionally in combination with an immunotherapy, are administered to the subject such that there is an overlap in the bioactive period of the one or more additional therapeutic agents and the inhibitors of the non-canonical NFkB pathway, e.g., of NIK, as described herein, and/or the optional immunotherapy, in the subject.

In some embodiments, the subject can be administered the at least one inhibitors of the non-canonical NFkB pathway, e.g., of NIK, as described herein, optionally in combination with an immunotherapy, over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of inhibitors of the non-canonical NFkB pathway, e.g., of NIK, as described herein, and/or optional immunotherapy, (and/or one or more additional therapeutic agents) administered to the subject and can also adjust (e.g., increase or decrease) the dosage or frequency of administration of at least one inhibitors of the non-canonical NFkB pathway, e.g., of NIK, as described herein, and/or optional immunotherapy, (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

In some embodiments, in addition to or as an alternative to the inhibitor of the non-canonical NFkB pathway, the methods can include targeted intratumoral delivery of IL-12 encoding plasmids (e.g., as described in Daud et al., 2008) in combination with an immunotherapy.

### EXAMPLES

The invention is further described in the following examples, which do not limit the scope of the invention described in the claims.

Experimental Model and Subject Details

Mice

All animals were bred and housed under specific pathogen free conditions at the Massachusetts General Hospital. Experiments were approved by the MGH Institutional Animal Care and Use Committee (IACUC) and were performed in accordance with MGH IACUC regulations. The following mouse strains were used in this study: Female C57BL6/J mice (8-12 week old) were purchased from Jackson Laboratories (Bar Harbor, Me.). GREAT (IFN-γ-IRES-eYFP Cat #017581), IL-12p40-IRES-eYFP (Cat #006412), CD11c-cre (Cat #007567), Ifngr1^{fl/fl} (Cat #025394), and Zbtb46-DTR (Cat #025394) were obtained from Jackson Laboratories.

Human Samples

Fresh tumor specimens were obtained from 6 adult cancer patients undergoing tumor resections at University Hospital Basel, Switzerland. Tissues were used for in vitro re-stimulation and analysis. The study was approved by the local Ethical Review Board (Ethikkommission Nordwestschweiz) and University Hospital Basel, Switzerland. All patients consented in writing to the analysis of their tumor samples.

| Patient | Gender | Health Status | Age |
|---|---|---|---|
| BS-661 | M | Cancer | 55 |
| BS-728 | M | Cancer | 77 |
| LA-061 | N/A | Cancer | 73 |
| BS-705_T | M | Cancer | 74 |
| BS-698_T | F | Cancer | 78 |
| BS-469_T | M | Cancer | 83 |

ImmunoPulse IL-12 treated tumor tissue samples were obtained from 19 melanoma patients from clinical trial NCT01502293. All biopsies were from University of California, San Francisco Medical Center-Mt. Zion, San Francisco, and Huntsman Cancer Institute, Salt Lake City, Utah, and were approved by each organization's institutional review board.

| Patient | Gender | Disease Status | Age |
|---|---|---|---|
| 1 | M | Stage III c | 66 |
| 2 | M | Stage III b | 88 |
| 3 | M | Stage IV M1c | 80 |
| 4 | F | Stage III c | 56 |
| 5 | M | Stage IV M1a | 65 |
| 6 | F | Stage III b | 89 |
| 7 | M | Stage IV M1a | 59 |
| 8 | M | Stage III c | 56 |
| 9 | M | Stage III c | 55 |
| 10 | M | Stage IV M1a | 63 |
| 11 | M | Stage IV M1a | 56 |
| 12 | M | Stage IV M1a | 44 |
| 13 | M | Stage IV M1a | 82 |
| 14 | M | Stage IV M1b | 74 |
| 15 | M | Stage IV M1b | 88 |
| 16 | M | Stage IV M1c | 58 |
| 17 | M | Stage III c | 61 |
| 18 | M | Stage III c | 59 |
| 19 | M | Stage III b | 65 |

Tumor Models

MC38 tumor cell lines were obtained from Dr. Mark Smyth (QIMR Berghofer). MC38 cells were implanted at 2×10⁶ cells in the flank. B16F10 cell lines were obtained from ATCC. B16F10 cells were implanted intradermally at 0.5×10⁶ cells in the flank. All tumor models were allowed to grow for one week prior to therapy. Tumor sizes were approximately 75 mm³ before treatment initiation, and starting tumor volumes were normalized between treatment groups. Percent tumor changes were calculated as percent difference of mouse tumor volume from pre-treatment baseline, measured using digital caliper. Lung seeding B16F10 models received 0.5×10⁶ cells intravenously and were allowed to grow for 10 days from the point of implantation. Mouse tumors were allowed to grow to a maximum of 2 cm in diameter, or until tumor ulceration occurred. These were considered as endpoints for survival experiments in accordance with MGH IACUC regulations.

Method Details

Immunotherapy Treatment and Cytokine Modulation

Tumor bearing mice, with a tumor size of approximately 75 mm³, were treated with 200 μg of aPD-1 and/or 100 μg of aCD40 intraperitoneally for immunotherapy studies. For combination treatment studies, both aPD-1 and aCD40 were administered at the same time. For IL-12 neutralization studies, mice were dosed with 500 μg of anti-IL-12p40 Clone 17.8 daily for 5-7 days following aPD-1 therapy. Neutralization of IFN-γ in vivo was performed by administering 1 mg of anti-IFN-γ Clone XMG1.2 initially with 500 μg of anti-IFN-γ dosed daily intraperitoneally for days 1-3. The cIAP1/2 inhibitor AZD5582 (Hennessy et al., 2013) was purchased from Selleck Chem and was resuspended in sterile saline. Mice received a single dose of AZD5582 at 10 mg/kg, intraperitoneally. For IL-12 supplementation studies, recombinant IL-12 (1 μg in 100 μL saline) was delivered peritumorally and intraperitoneally, half dose each, for 5 consecutive days when indicated.

Tumor Re-Challenge

Long-term surviving mice from aPD-1 and aCD40 combination therapy were re-challenged with either MC38 or B16F10 tumors at 50 days following primary tumor rejection. MC38 and B16F10 re-challenge doses were 2×10⁶ cells and 0.5×10⁶ cells respectively in the contralateral flank. Naive C57BL/6J mice were implanted alongside re-challenge mice, and these mice were monitored for tumor growth for 2 weeks following implantation.

Bone Marrow Chimeras

For bone marrow chimera studies, recipient C57BL/6J mice were irradiated (10 Gray dose) in one session, and mice were injected intravenously with 5×10⁶ or 3×10⁶ whole bone marrow cells from B6(Cg)-Zbtb46^{tml(HBEGF)Mnz}/J (Zbtb46-DTR) or B6N.129-Map3k14^{tmlRds}/J (Nik^{-/-}) respectively. Control mice were irradiated and re-constituted with C57BL/6J whole bone marrow (5×10⁶ cells). Mice were then left to reconstitute for 8 weeks before tumor growth experiments. Mice receiving diphtheria toxin (DT) (Sigma-Aldrich) were dosed at 10 ng of DT per gram of body weight to initiate depletion and then maintained at 4 ng of DT per gram of body weight every 3 days following initial depletion.

Single Cell RNA Sequencing

MC38 tumors were implanted into the flanks of C57BL6/J mice and allowed to grow for 7 days before immunotherapy treatment. Mice were untreated or aPD-1 treated. Tumors were harvested 3 days after initiation of therapy. Tumors were digested using collagenase II (Worthington) and CD45⁺ cells were sorted from single cell suspensions using a BD FACSAria sorter. Cells were manually counted with a hemocytometer and trypan blue viability stain, and 3132 cells from the control treated and 8178 cells from the aPD-1 treated tumors were recovered directly in PBS with 0.04% BSA (400 μg/ml) without centrifugation and kept on ice. Live cells were single cell sorted into GEMS (Gel Bead in EMulsion) using the 10× Genomics Chromium system provided by the HMS Biopolymers core. GEMS were processed and libraries were prepared according to the Chromium Single Cell 3′ Reagent Kit v2 User guide (10× Genomics). Library QC was done by the HMS Biopolymers core and the libraries were sequenced on an Illlumina NextSeq at an average of 29,000 reads per cell. In total, 4095 cells (1154 untreated and 2941 aPD-1 treated cells) passed QC and were sequenced. 10× Cell Ranger 2.1.0 software was used for generation of fastq files and gene-barcode matrices. Loupe Cell Browser 2.0.0 and the Seurat R package (Satija et al., 2015) and SPRING (Weinreb et al., 2017) were used for clustering and analysis.

Parabiosis

CD45.3 and B6.129-Il12b^{tmlLky}/J (IL-12 reporter) mice were placed under anesthesia (2% isoflurane) shaved on their sides and elbows and knees were stitched together with a black monofilament nylon suture (Ethicon). Animals were provided with buprenorphine as an analgesic for 3 days following surgery. After a 3 week recovery period, both mice from the parabiotic pair were challenged with MC38 tumors on the outer flank. Tumors were allowed to grow for 7 days before treatment with aPD-1 immunotherapy, and tumors were harvested 2 days following immunotherapy to analyze IL-12 dendritic cell populations by flow cytometry.

FTY720 Treatments

Mice were implanted with MC38 tumors in the flank and cohorts of mice were sorted into groups of similar tumor size before treatment initiation. Tumors were allowed to grow for 7 days before treatments. Mice were treated or not with 1.25 mg/kg of FTY720 (Cayman Chemical) i.p. 2 hours before aPD-1 treatment, and were maintained daily on 1.25 mg/kg FTY720 throughout the duration of the experiment. Blood from mice was used to confirm lymphocyte trafficking defects.

Flow Cytometry—Mouse

Tumor tissue or tumor draining lymph nodes were isolated from mice and minced using surgical scissors. Tissues were then digested using 0.2 mg/ml Collagenase II (Worthington) in RPMI 1640 media (CellGro) at 37° C. for 30 minutes and then strained through a 40 μm filter (BD Falcon). Cell suspensions were incubated with Fc Block TruStain FcX Clone 93 (Biolegend) in PBS containing 0.5% BSA and 2 mM EDTA before staining with fluorochrome labeled antibodies. Antibodies against CD11b (M1/70, Biolegend), CD8a (53-6.7, Biolegend), CD45 (30-F11, BD), F4/80 (BM8, Biolegend), CD11c (N418, Biolegend), MHC II I-A/I-E (M5/114.15.2, Biolegend), CD103 (2E7, Biolegend), IFN-γ (XMG1.2, Biolegend) were used for marker staining. 7AAD viability staining was used to exclude dead cells from analysis. Samples were run on a LSR II flow cytometer and analyzed using FlowJo software (Treestar). For intracellular cytokine staining, samples were incubated for 5 hours with GolgiPlug (BD) at 1 μl per ml of culture media. Cells were then surface stained and then fixed and permeabilized using the BD Cytofix/Cytoperm kit (BD) according to manufacturer's protocol and stained for intracellular cytokines.

Intravital Imaging

Interferon gamma reporter (IFN-γ-eYFP) or IL-12p40 reporter (IL12p40-eYFP) mice were anesthetized and dorsal skin-fold window chambers were installed as previously described (Thurber et al., 2013) and mice were treated with analgesic (Buprenorphine 0.1 mg/kg/day) for 3 days following chamber implantation. Twenty-four hours after window implantation, MC38-H2B-mApple cells (2×10⁶ in 20 μl) were injected in the fascia layer. Pacific Blue-dextran nanoparticle (containing 1 nmol Pacific Blue dye) was injected 1 week after tumor implantation for macrophage labeling. On the next day, Pacific Blue-dextran (containing 37 μg dextran and 56 nmol Pacific Blue dye) for vascular labeling was delivered via a 30-gauge catheter inserted in the tail vein of the anesthetized mouse (2% isoflurane in oxygen). Anesthetized mice were kept on a heating pad kept at 37° C., vital signs monitored and mice were imaged using an Olympus FluoView FV1000MPE confocal imaging system (Olympus America). A 2× air objective XL Fluor 2×/340 (NA 0.14; Olympus America) was used to select regions near tumor margins and tumor vasculature by an operator blinded to treatment conditions. Higher magnification Z-stack images were acquired using a XLUMPLFL 20× water immersion objective (NA 0.95; Olympus America) with 1.5× digital zoom. Sequential scanning (5 μm slices) with 405, 473, 559, and 635 nm lasers was performed using voltage and power settings that were optimized using fluorescence minus-one control mice prior to time lapse acquisition. DM405/473/559/635 nm dichroic beam splitters (SDM473, SDM560, and SDM 640) and emission filters (BA430-455, BA490-540, BA575-620, BA575-675) were sourced from Olympus America. For time lapse acquisitions, a total frame interval of 133 seconds was acquired at non-overlapping coordinates. For CD8⁺ cell depletions, 200 μg of aCD8 was delivered 24 hours prior to aPD-1. Unlabeled antibodies were used with the exception of specific cases where AF647-aPD-1 mAb or AF647-aCD40-mAb were delivered for drug distribution studies. Fluorochrome labeled antibodies were delivered at the same dose as unlabeled antibodies.

Isolation of Tumor-Infiltrating Lymphocytes and In Vitro Restimulation

MC38 tumors were digested into single cell suspensions similar to tissue processing for flow cytometry analysis and were passed through a 40 μm filter. Cells were then labeled using the Miltenyi CD8a T cells enrichment kit (Miltenyi Biotec) and isolated using magnetic sorting according to manufacturer's protocols. Tissue culture plates were coated with anti-CD3ε and anti-CD28 at a concentration of 10 μg/ml and 5 μg/ml respectively in PBS for 12 hours, and excess antibody was aspirated before T cell addition. IL-12 was added into culture media at a concentration of 20 ng/ml. Cells were stimulated for 72 hours before addition of GolgiPlug for 5 hours for intracellular cytokine staining.

Tissue Isolation and Quantitative PCR

Fresh MC38 tumor tissue (20-30 mg) from WT C57BL6/J aPD-1 treated mice, WT C57BL6/J aPD-1/aIFN-γ treated mice, aPD-1 treated Ifngr1^{fl/fl} mice, or aPD-1 treated Cd11c-cre Ifngr1^{fl/fl} mice, were finely minced using surgical scissors and lysed in RLT lysis buffer (Qiagen) and frozen. Samples were then thawed and RNA was extracted using the Qiagen Mini RNA extraction kit, and reverse transcribed using the High-Capacity cDNA kit (Thermo Fisher Scientific). Quantitative PCR was performed using Il12p40 Taqman Gene Expression probes (Thermo Fisher Scientific) and referenced to HPRT expression using 10 ng of cDNA per sample. The ΔΔCT method was used to quantitate Il12p40 expression across samples.

Human Studies

We performed two human studies to address the following questions: the first study aimed to define whether IL-12 delivery into tumors can enhance antitumor T cell signatures in vivo (ImmunoPulse, tavokinogene telseplasmid, IL-12 studies); the second study assessed whether IL-12 can activate tumor-infiltrating CD8⁺ T cells directly (IL-12 ex vivo studies), as detailed below.

ImmunoPulse IL-12 studies: Tumor biopsies from 19 melanoma patients enrolled in an ongoing clinical trial (NCT01502293) were used to assess whether intratumoral treatment with ImmunoPulse IL-12, a plasmid electroporation method that delivers IL-12 directly to tumors (Daud et al., 2008), induced a cytolytic immune signature within tumors. Patients presented with stage IIIB, IIIC, or IV M1a melanoma and with at least one lesion≥0.3 cm×0.3 cm in longest perpendicular diameters that was accessible for electroporation; patients may have had prior chemotherapy or immunotherapy (excluding prior therapy with IL-12 or gene therapy) but must have stopped treatment at least 4 weeks prior to study enrollment. All biopsies were from University of California, San Francisco Medical Center-Mt. Zion, San Francisco, and Huntsman Cancer Institute, Salt Lake City, Utah. Skin tumor tissue was isolated at two time points: first on the day of screening and then either on week 4 (15 of 19 patients (78.9%)), 6 (3 of 19 patients (15.8%)) or 12 (1 of 19 patients (5.3%)) after treatment began. The same lesions were biopsied pre-treatment and post-treatment when possible, regardless of time point. If no matched post-treatment lesions were available, week 4 biopsies from unmatched lesions were used (12 of 19 lesions (63.2%) were matched, 7 of 19 lesions (36.8%) were unmatched). Biopsies were fixed in PAXgene tissue fixative (PreAnalytiX, Hombrechtikon, Switzerland) and embedded in paraffin at Cureline (Brisbane, Calif.). 8×10 micron tissue curls were used for RNA extraction via RecoverAll™ Total Nucleic Acid Isolation Kits (ThermoFisher Waltham, Mass.) according to manufacturer's protocol. If necessary, RNA was concentrated using RNA Clean & Concentrator-5 kits according to manufacturer's protocol (Zymo Research Irvine, Calif.). Up to 100 ng of RNA was run on NanoString's PanCancer IO360™ beta version (NanoString Technologies Seattle, Wash.). Analysis was completed using NanoString's nSolver analysis software 3.0 pack. Data were normalized to control genes. Data were excluded if binding density, positive controls, or normalization factors were outside of the acceptable ranges set by NanoString. Post-treatment signals from selected genes were normalized to matched pre-treatment sample signals and plotted as a fold change relative to pre-treatment gene expression data.

IL-12 ex vivo studies: Fresh tumor resections from six cancer patients undergoing surgical treatment at University Hospital Basel, Switzerland were used to assess whether IL-12 can directly activate human tumor-infiltrating CD8⁺ T cells upon isolation of these cells ex vivo. Tumor tissue (two lung adenocarcinomas, three squamous cell carcinomas and one synovial sarcoma) was collected from six different patients undergoing primary surgical treatment between November 2015 and November 2017. The study was approved by the local Ethical Review Board (Ethikkommission Nordwestschweiz) and all patients consented in writing to the analysis of their tumor samples. The solid tumor lesions were mechanically dissociated and enzymatically digested using accutase (PAA), collagenase IV (Worthington), hyaluronidase (Sigma) and DNAse type IV (Sigma), directly after excision. Single cell suspensions were prepared and cryopreserved in liquid nitrogen in 90% fetal calf serum (FCS, Brunschwig Pan Biotech) and 10% dimethylsulfoxide (DMSO, Sigma) until further usage. Thawed tumor digests were stained with the appropriate fluorochrome-coupled antibodies in PBS with 2% FCS and sorted for CD8⁺ T cells by flow cytometry using a BD SorpAriaIII. Sorting purity was measured by reanalyzing the sorted cells and always reached >95% purity. Cells were rested at 37° C., 5% CO₂ in 96 well plates in supplemented RPMI medium (Sigma, supplemented with 10% heat-inactivated and tested FCS, 1 mM pyruvate, 2 mM glutamine, 1% penicillin and streptomycin, 1% non-essential amino acids) for 18 hours, and further stimulated with 10 ng/ml recombinant human IL-12p70 (PeproTech) and/or 0.5 μg/ml OKT3 anti-CD3 antibody (UltraLEAF Purified, Biolegend) in supplemented RPMI medium and incubated for 3 days at 37° C., 5% CO₂. IFN-γ secreted by these cultures was then measured by enzyme-linked immunosorbent assay according to the instructions by the manufacturer (BD, OptEIA human IFN-γ ELISA set). The following anti-human mAbs were used: CD3 PE (clone SK7, eBioscience); CD4 BV711 (clone SK3, BD); CD8 FITC (clone SK1, eBioscience); CD11b PerCP eFluor710 (clone ICRF44, eBioscience), CD11c PerCP eFluor710 (clone 3.9, eBioscience); CD14 PerCP-eFluor710 (clone 61D3, Biolegend); CD19 PerCP-Cy5.5 (clone SJ25C1, Biolegend); CD45 APC-H7 (clone 2D1, BD Pharmingen); CD56 APC (clone AF12-7H3, Miltenyi).

### Quantification and Statistical Analysis

Image Processing

Images were Z-projected, cropped, and de-speckled for clarity using FIJI running ImageJ version 2 (6). For quantification, raw Z stack images were processed using rolling ball background subtraction, Renyi Entropy thresholding, and cell counting macros run through customized Java scripts in the FIJI environment. TAM and tumor vessels were segmented by pixel size and shape exclusion parameters. Cell number divided by area were reported relative to baseline prior to treatment. The Manual Tracking Plugin was used in FIJI for cell tracking. The slope of the regression function fitted to the mean displacement plot for each cell calculated to derive the cell motility coefficients (M), according to the following formula: M=d²/4t, where d is displacement from origin at time t.

Statistical Analysis

Flow and imaging data were collected using FlowJo Version 10.4 and the FIJI package of ImageJ running version 1.51s. This and other primary data was collected and organized using Microsoft Excel (version 14.6.3). All statistical analyses were performed using Graphpad Prism Version 7. Mouse cohort sizes were pre-determined using power analyses, as reported previously (Arlauckas et al., 2017). Values reported in figures are expressed as the standard error of the mean, unless otherwise indicated. For normally-distributed datasets, we used 2-tailed Student's t test and one-way ANOVA followed by Bonferroni's multiple comparison test. When variables were not normally distributed, we performed non-parametric Mann-Whitney or Kuskal-Wallis tests. For survival analysis, p-values were computed using the Log Rank test. p-values>0.05 were considered not significant (n.s.), p values<0.05 were considered significant. *p-value<0.05, **p-value<0.01, ***p-value<0.001, ****p-value<0.0001.

Data and Software Availability

Raw data for single cell RNA sequencing from sorted CD45⁺ cell populations from MC38 tumors can be found at the Gene Expression Omnibus Repository (GEO). The accession number for control (untreated) samples is GSM3090155. The accession number for aPD-1-treated samples is GSM3090156.

### Example 1. Successful aPD-1 Treatment Triggers Endogenous IFN-γ and IL-12 Responses within Tumors

To image key readouts of immunotherapy function, we assessed IFN-γ and

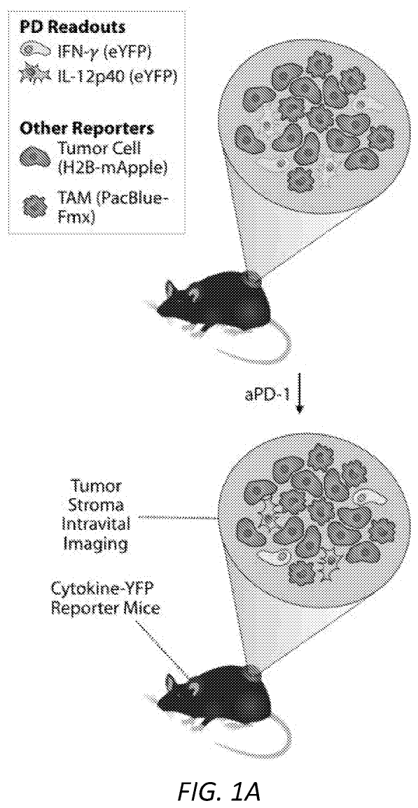

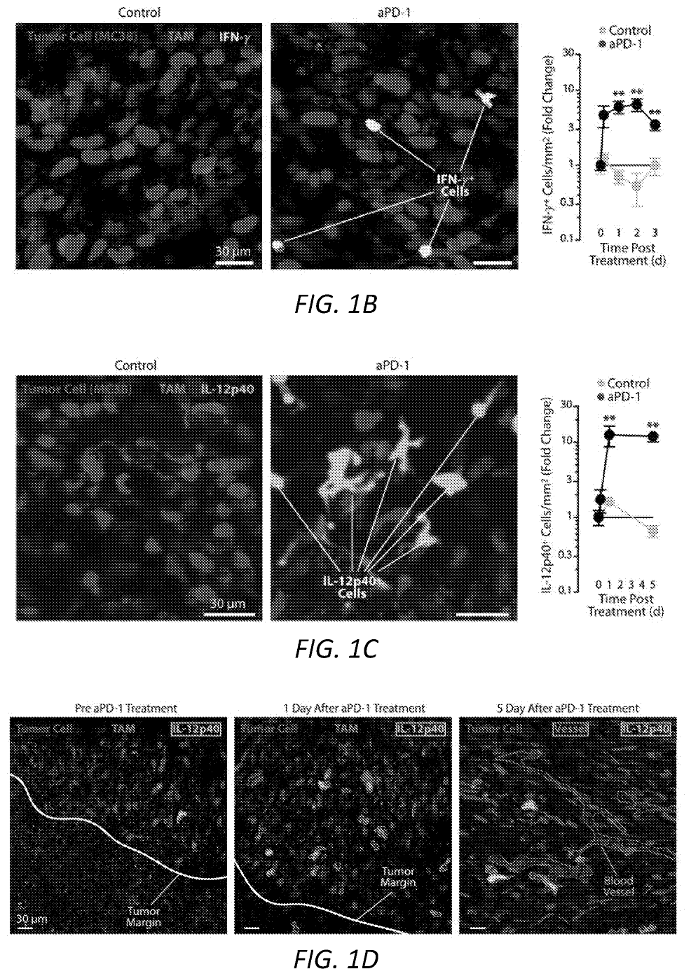

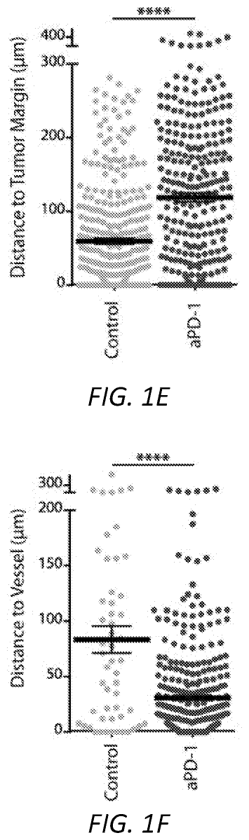

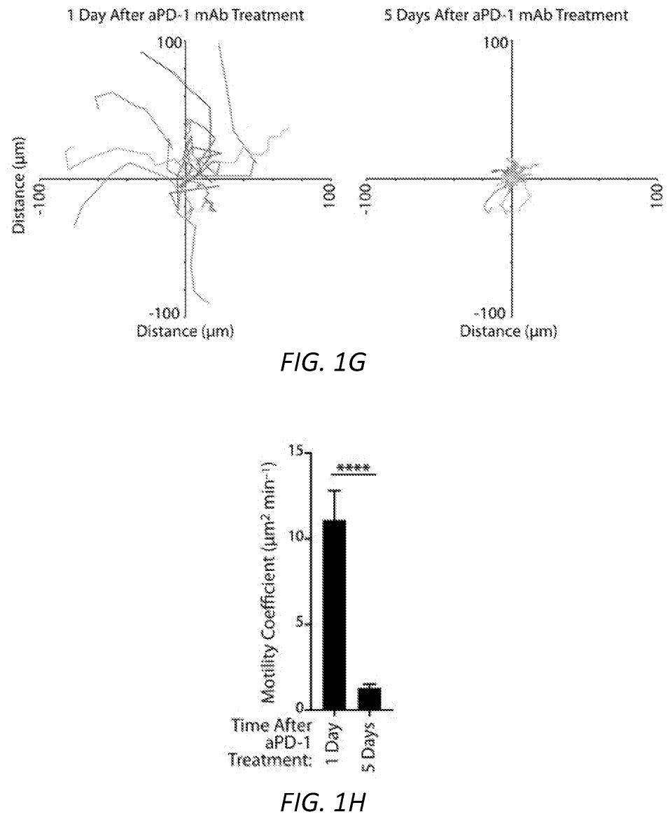

IL-12p40, a protein subunit of IL-12 and IL-23, production using IFN-γ-internal ribosome entry site-yellow fluorescent protein (IFN-γ-IRES-YFP) and IL-12p40-IRES-YFP reporter mice, hereafter referred to as IFN-γ-eYFP and IL-12p40-eYFP, respectively (FIG. 1A). Intravital imaging detects YFP, which is expressed by cells that have turned on IFN-γ or IL-12p40 production (Reinhardt et al., 2015; Reinhardt et al., 2006). YFP remains detectable even after cytokine production is turned off, which makes intravital imaging a particularly useful tool to detect the activation of molecules with rapid on/off cycling, such as IFN-γ (Slifka et al., 1999). We tracked IFN-γ and IL-12p40 in vivo during rejection of aPD-1 treatment-sensitive MC38 tumor cells, which were labeled with H2B-mApple. We also tracked macrophages, which were tagged with Pacific-blue-dextran nanoparticles (Weissleder et al., 2014), as these cells are often abundant in tumors (Engblom et al., 2016).

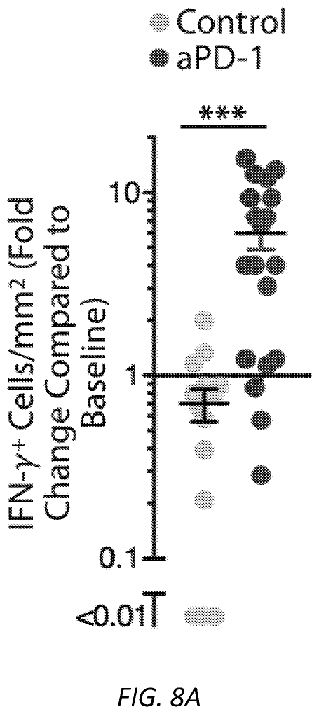

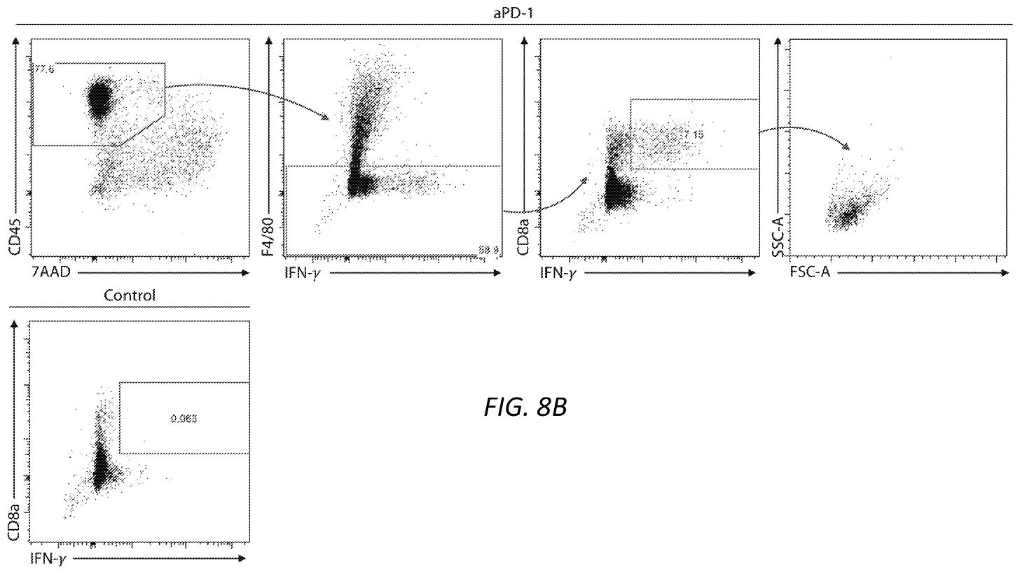

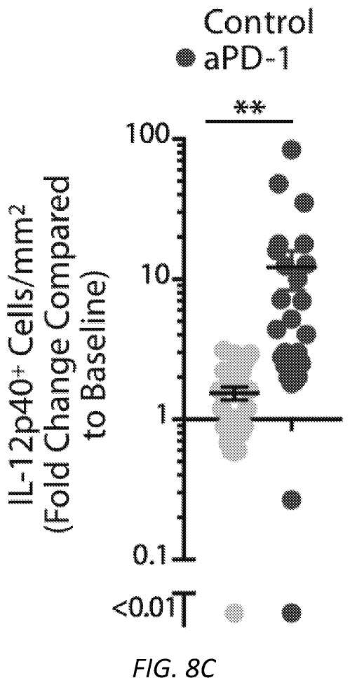

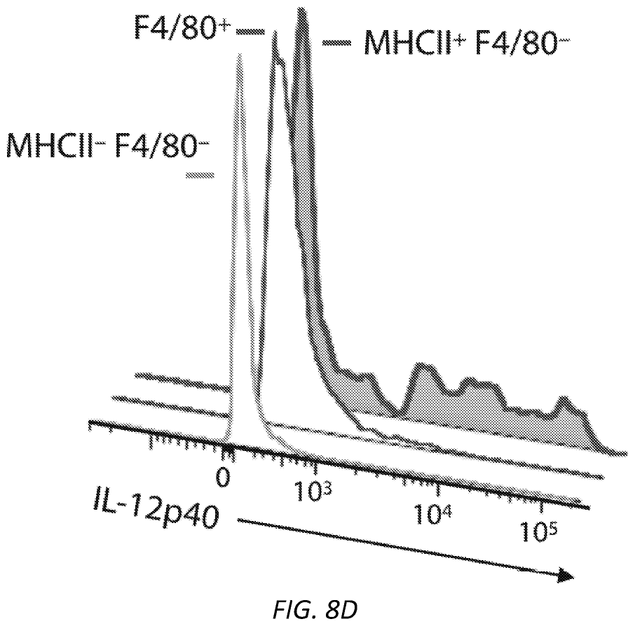

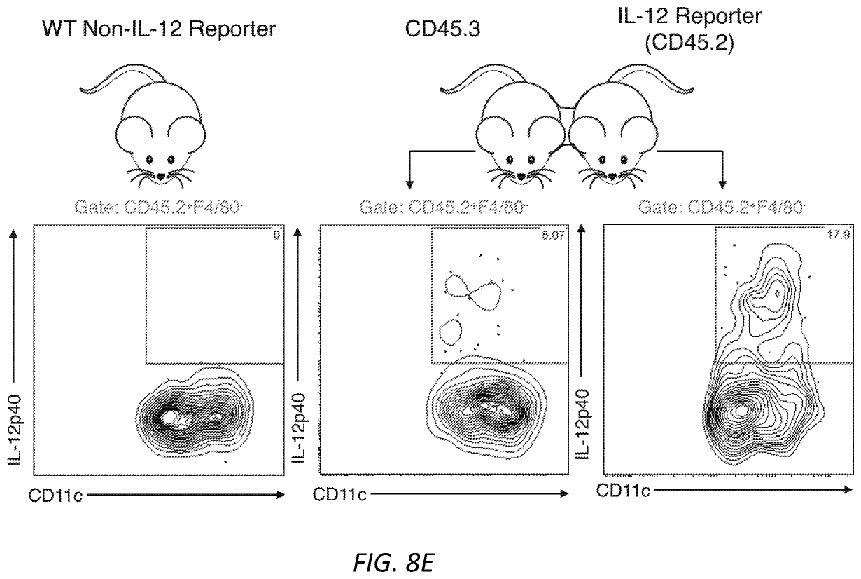

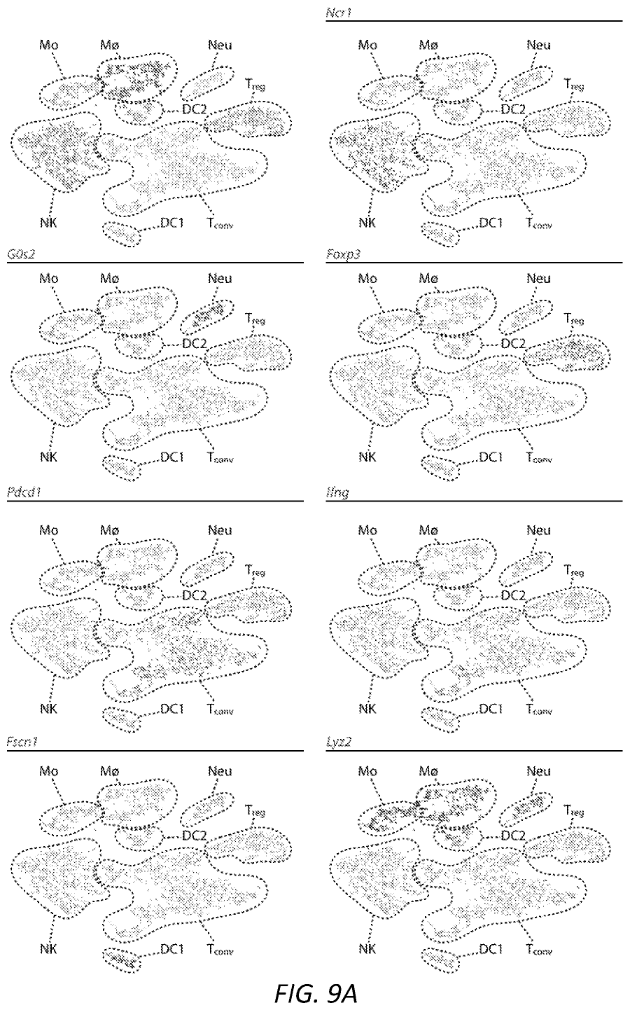

Intravital imaging of the tumor microenvironment revealed a 6.0±1.1 (mean±SEM) fold expansion of IFN-γ-eYFP⁺ cells one day after a single aPD-1 injection; this increase was sustained for up to 3 days post treatment (FIGS. 1B and 8A). IFN-y-eYFP⁺ cells accumulated within the tumor stroma and were mostly CD8⁺ T cells (FIG. 8B). Intravital imaging further revealed a 12.1±3.7 fold increase of IL-12p40-eYFP⁺ cells on day one post treatment, which persisted for at least five days (FIGS. 1C and 8C). IL-12p40-eYFP⁺ cells displayed a branched morphology (mean circularity index: 0.54±0.4), suggesting they were DCs. In comparison to the few IL-12⁺ cells detected before aPD-1 treatment, those present after treatment accumulated in deeper regions of the tumor (FIG. 1D, E) and closer to vessels (FIG. 1F). The ability for IL-12⁺ cells to accumulate within tumors was supported by the real-time imaging observation that these cells were motile one day after aPD-1 treatment (motility coefficient: ˜10 μm²/min; FIGS. 1G-H) and much less so on day five (<1 μm²/min; FIG. 1G-H). These findings indicate that aPD-1 delivery to tumors functionally impacts at least two non-overlapping cell populations, which respond differently to treatment: CD8⁺ T cells that activate the IFN-γ signaling pathway, and DC-like cells that turn on IL-12 production.

### Example 2. scRNAseq Shows DC-Restricted IL-12 Production

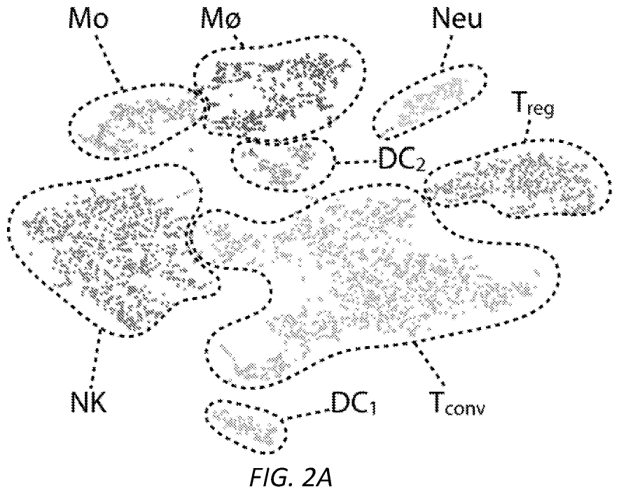

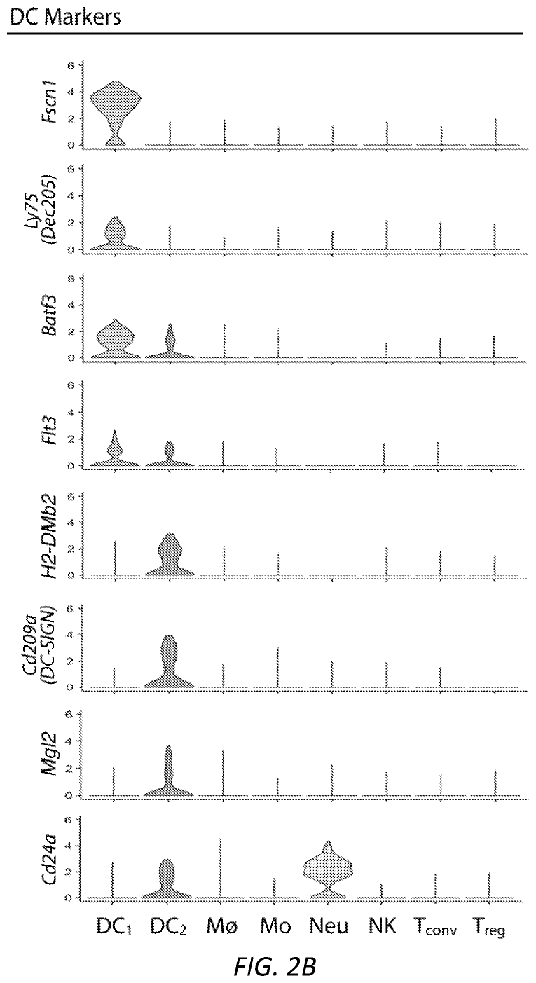

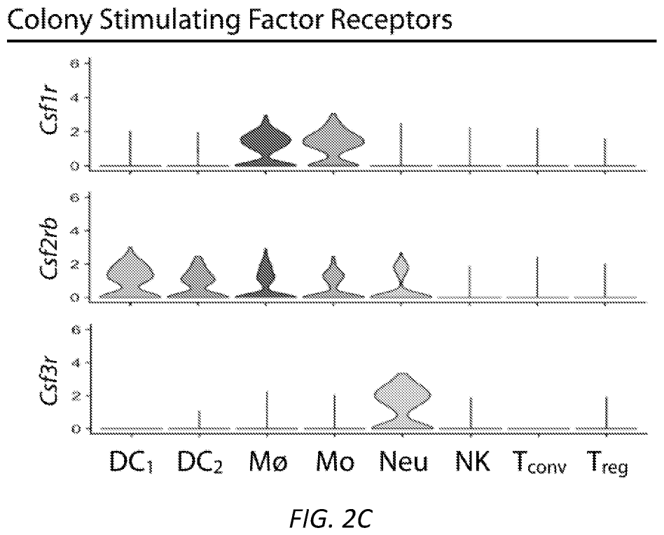

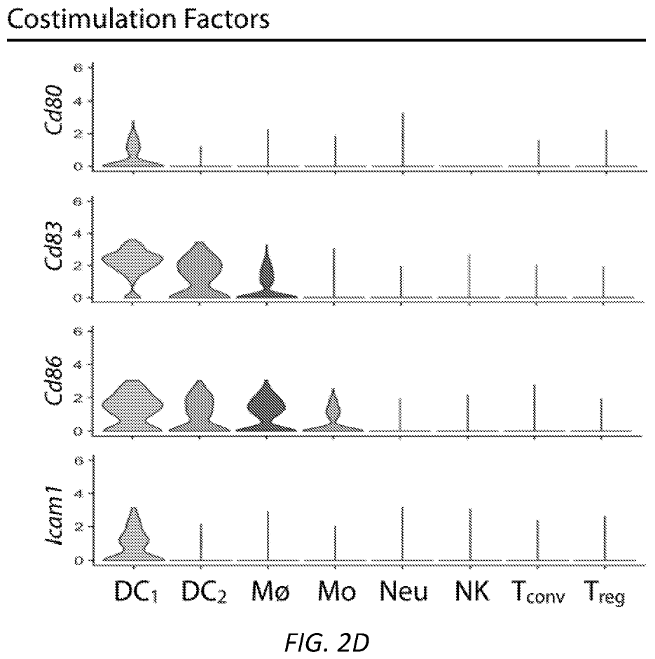

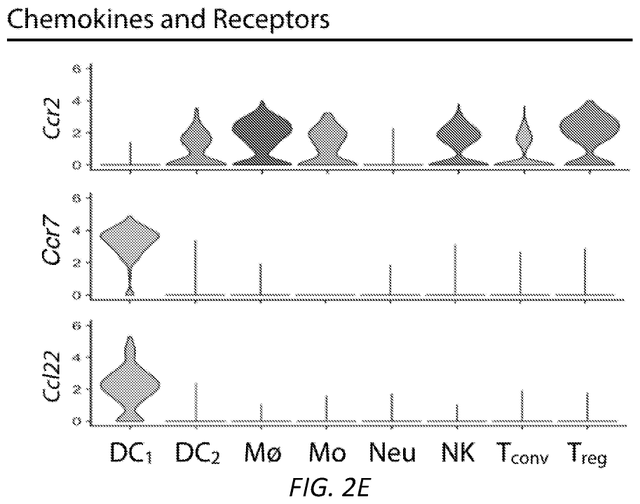

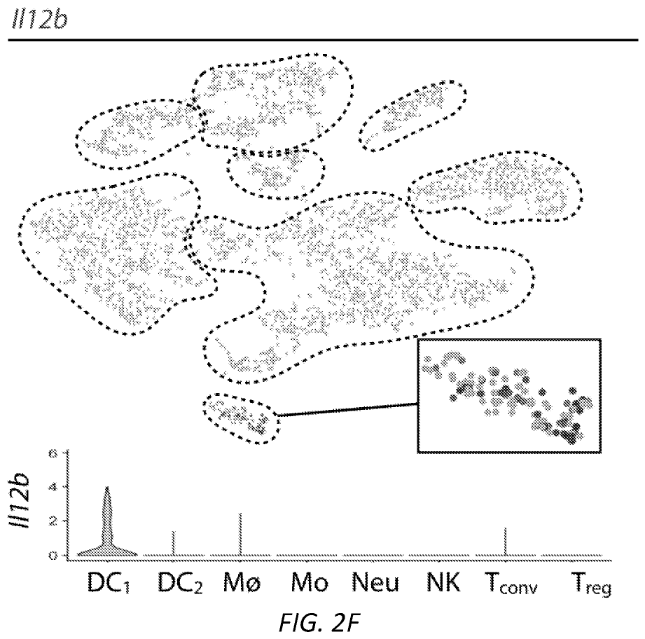

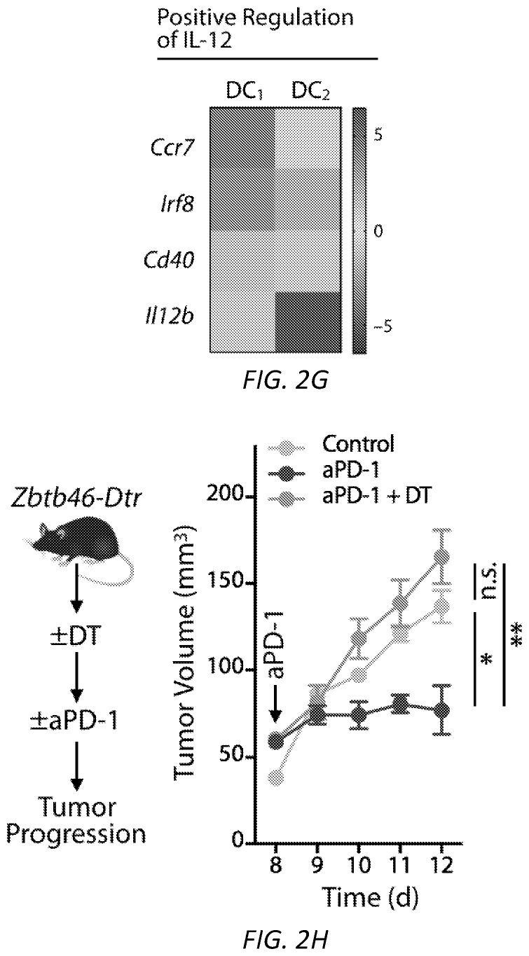

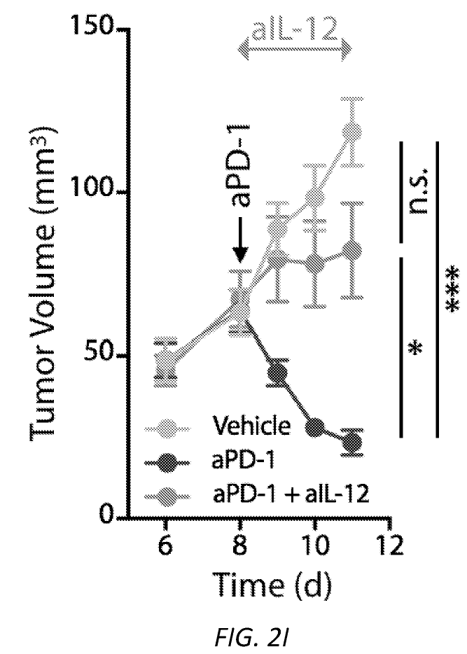

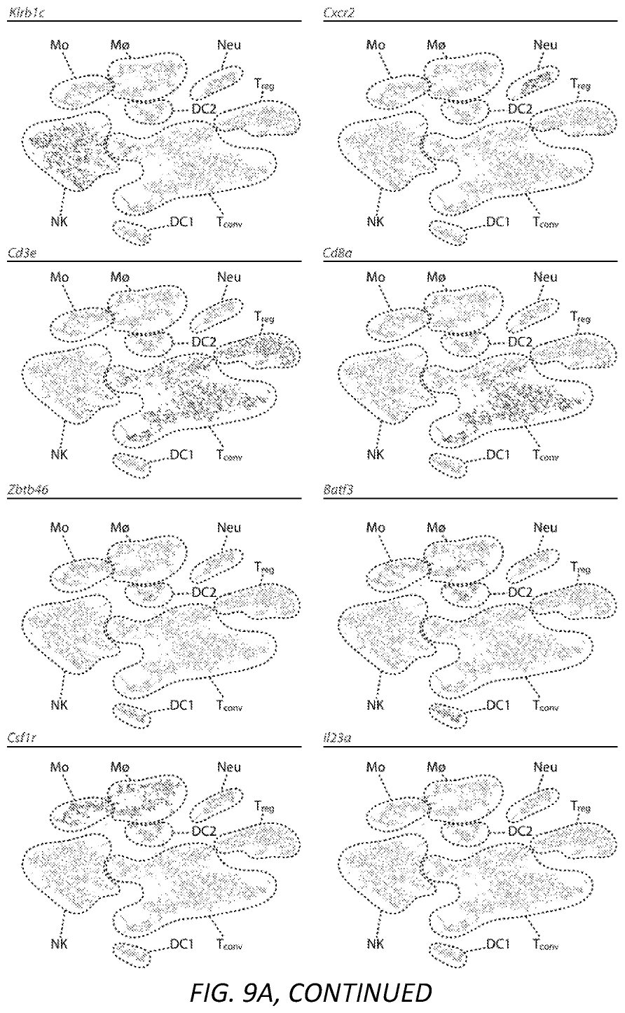

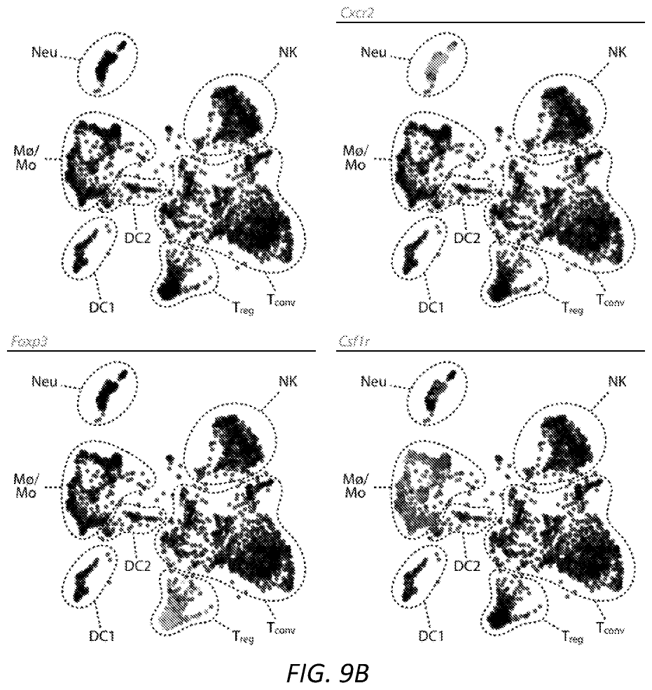

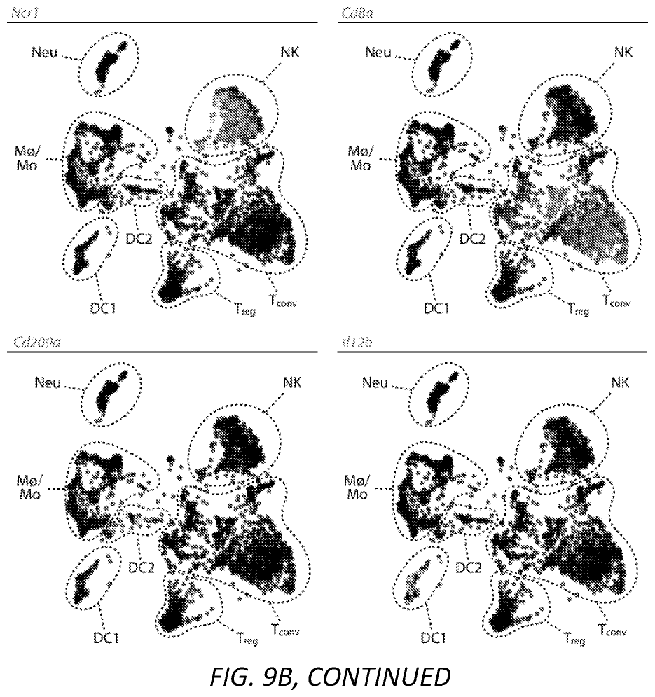

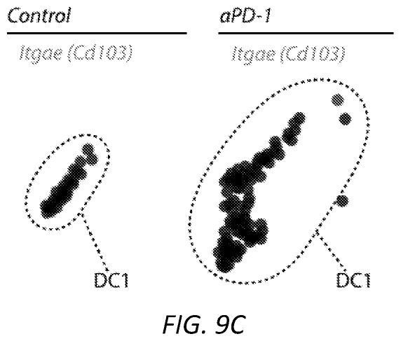

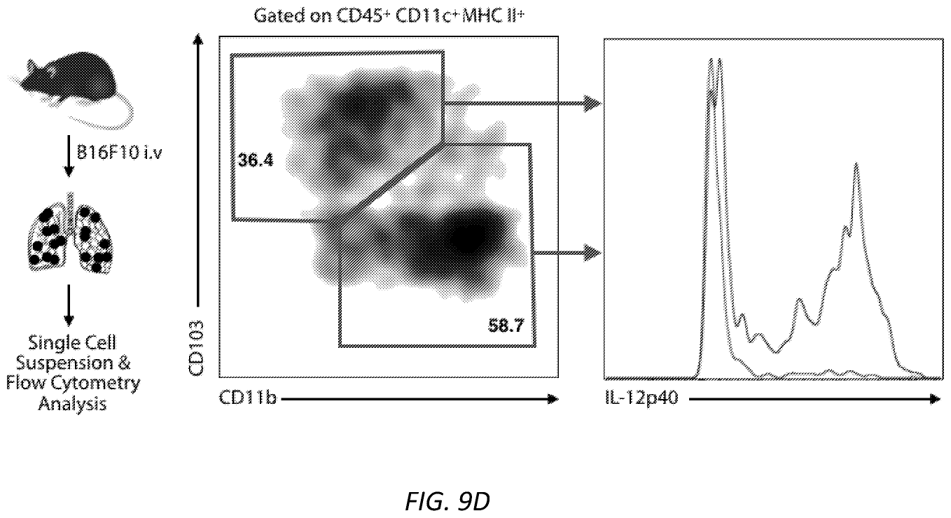

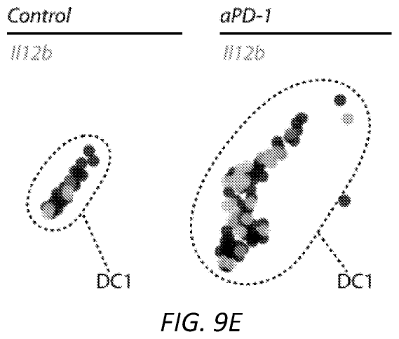

We next sought to further characterize the aPD-1-induced IL-12⁺ DC-like cells. Flow cytometry analysis confirmed these cells to be MHC class II⁺ F4/80⁻ (FIG. 8D), and parabiosis of tumor-bearing mice indicated that these cells could derive from a blood circulating precursor (FIG. 8E). To provide a more comprehensive and unbiased view of immunotherapeutic responses across the tumor immune microenvironment, including all myeloid cell types, we performed scRNAseq analysis on CD45⁺ cells isolated from untreated (n=1,154 cells sequenced) or aPD-1-treated (n=2,941 cells sequenced) tumors. All cells (n=4,095) were clustered into unbiased cell type classifications using the Seurat single cell analysis R package (Macosko et al., 2015). The cell clusters, visualized with t-stochastic neighbor embedding (t-SNE; FIG. 2A and FIG. 9A) or force-directed graph layouts (SPRING (Weinreb et al., 2017); FIG. 9B), identified the following populations: conventional T (Tconv) cells expressing Cd3e, regulatory T (Treg) cells expressing the transcription factor forkhead box P3 (Foxp3), natural killer (NK) cells expressing natural cytotoxicity triggering receptor 1(Ncr1) and killer cell lectin-like receptor subfamily B member 1c (Klrb1c), neutrophils (Neu) expressing C-X-C motif chemokine receptor 2 (Cxcr2) and G0/G1 switch 2 (G0s2), monocytes (Mo) and macrophages (Mø) expressing colony stimulating factor 1 receptor (Csf1r), and two DC subsets, referred to as DC1 and DC2.

Both DC1s and DC2s expressed the DC markers Batf3, Flt3, H2-Dmb2 and Zbtb46 (Meredith et al., 2012; Hildner et al., 2008;); DC1 expressed Fscn1 and Ly75 (DEC-205) whereas DC2s expressed CD209a (DC-SIGN), Mgl2 (CD301b) and Cd24a (FIG. 2B and FIG. 9B). Both DC subsets were largely negative for the macrophage colony-stimulating factor receptor Csf1r (FIG. 2C), although some DC2s expressed this receptor (FIG. 9A), similarly to a subset of intratumoral DCs previously reported (Broz et al., 2014). DC1s had higher expression of the granulocyte/macrophage colony-stimulating factor receptor Csf2rb compared to DC2s, and neither DC1s nor DC2s expressed the granulocyte colony-stimulating factor receptor Csf3r (FIG. 2C). Additionally, DC1s were enriched for the T cell co-stimulatory factors Cd80, Cd83, Cd86 and Icam1 (FIG. 2D), and DC and DC2s expressed distinct chemokines and chemokine receptors (FIG. 2E).

IL-12p40 (also known as IL12b) expression was contained exclusively within the DC1 population (FIG. 2F). Curating genes defined from gene ontology for positive regulation of IL-12 signaling and synthesis (GO:0045084; GO:0032735), we found that DC1s were enriched in IL-12-related production factors such as Cd40 and Irf8 (FIG. 2G). IL-12⁺ DCs in MC38 tumors did not express Itgae (the gene encoding the integrin CD103) (FIG. 9C), although previous studies identified CD103⁺ DCs as important cells for immune responses to tumors (Salmon et al., 2016; Spranger et al., 2015; Ruffell et al., 2014; Broz et al., 2014). This discrepancy may be due to tissue location, as we found that IL-12⁺ DCs expressed CD103 in lung tumor models (FIG. 9D). scRNAseq analysis confirmed the expansion of IL-12⁺ DCs after aPD-1 treatment (FIG. 9E). Collectively, these data demonstrate a distinct population of IL-12-producing DCs in the tumor microenvironment.

### Example 3. DCs and IL-12 are Relevant to aPD-1 Therapy

To assess whether DCs are relevant to aPD-1 treatment, we generated Zbtb46-DTR bone marrow chimeras (Meredith et al., 2012), which allowed us to deplete DCs selectively and do so after tumors were established, but before aPD-1 treatment was initiated. Mice lacking DCs failed to reject tumors in response to aPD-1 (FIG. 2H), indicating that these cells were required at the time when aPD-1-mediated tumor rejection occurs. To define whether IL-12 contributes to aPD-1 therapeutic efficacy, we studied DC-sufficient MC38 tumor-bearing mice that received aPD-1 in the presence or absence of neutralizing IL-12 mAbs. Mice in which IL-12 was neutralized failed to reject tumors, indicating that IL-12 production following aPD-1 treatment was necessary for achieving tumor control (FIG. 2I). Collectively, these data indicate that aPD-1 treatment induces IL-12 production by DCs, and that both DCs and IL-12 critically regulate aPD-1 treatment potency. The results accord with previous findings that tumor-infiltrating DCs can foster T cell immunity (Broz et al., 2014; Salmon et al., 2016) and immunotherapeutic responses (Alloatti et al., 2017), and here we show that DCs assist antitumor responses by providing cytokine support to the tumor immune microenvironment.

### Example 4. IFN-γ Sensing by DCs Controls IL-12 Production

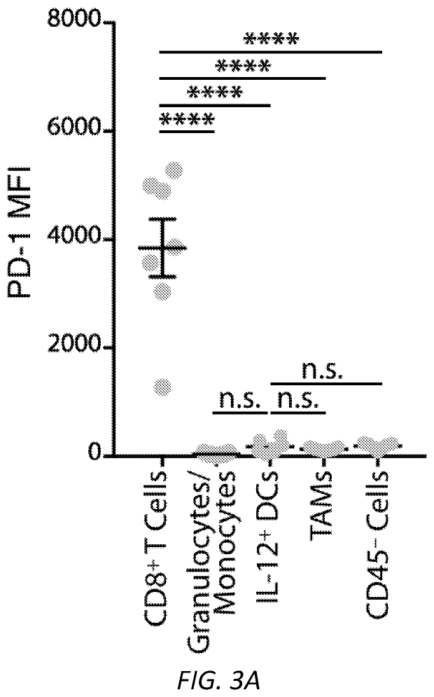

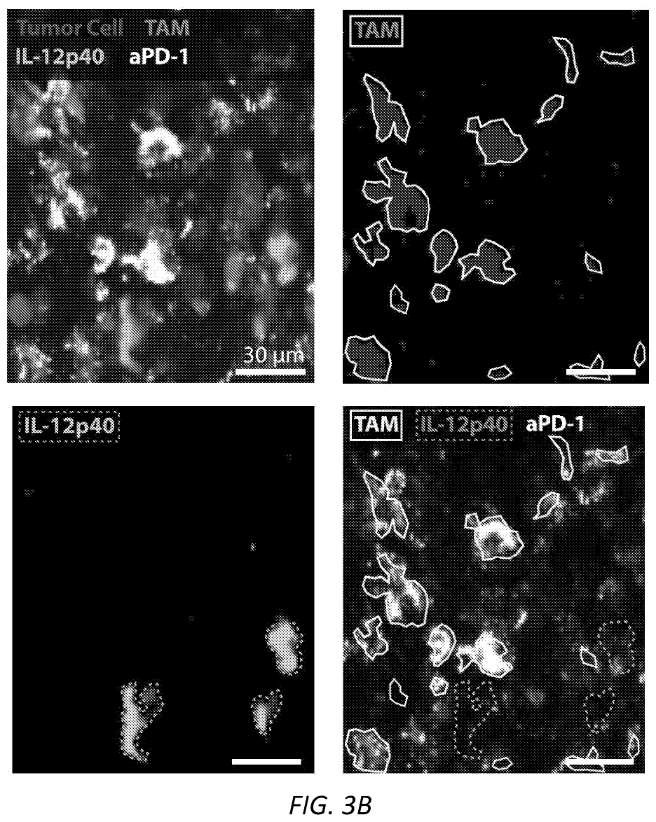

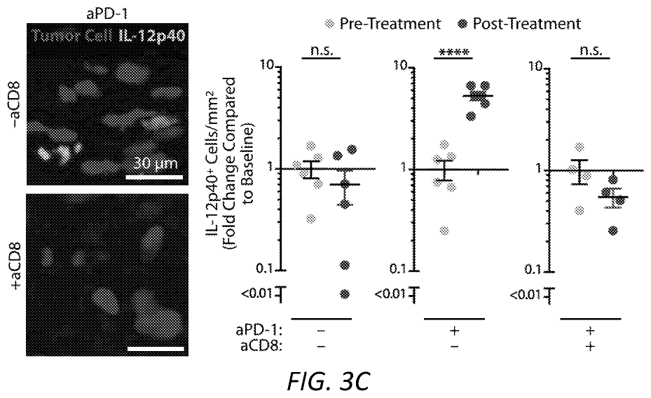

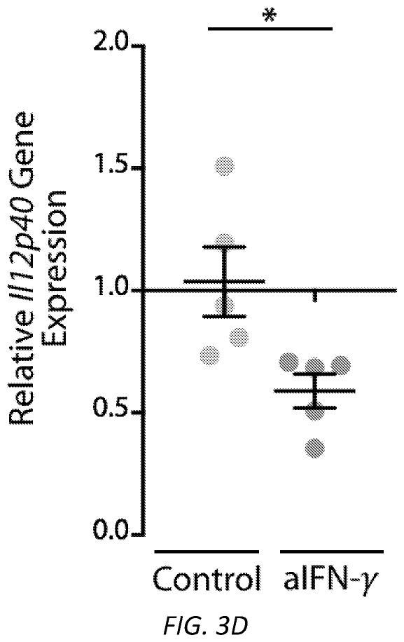

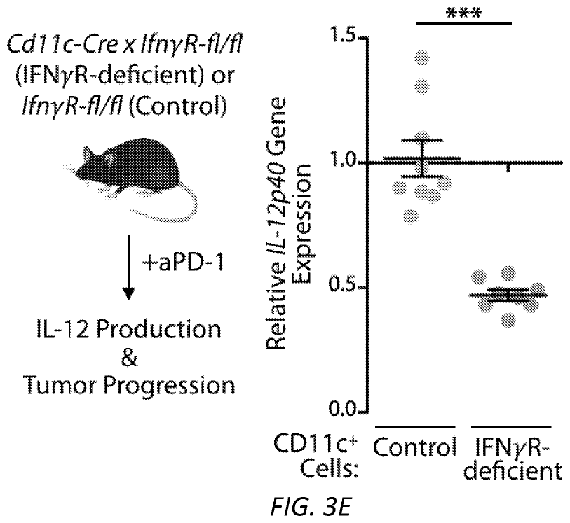

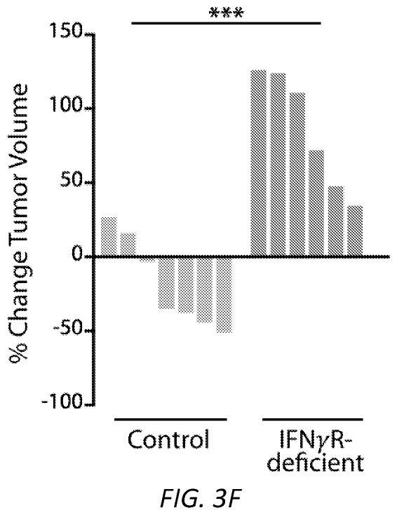

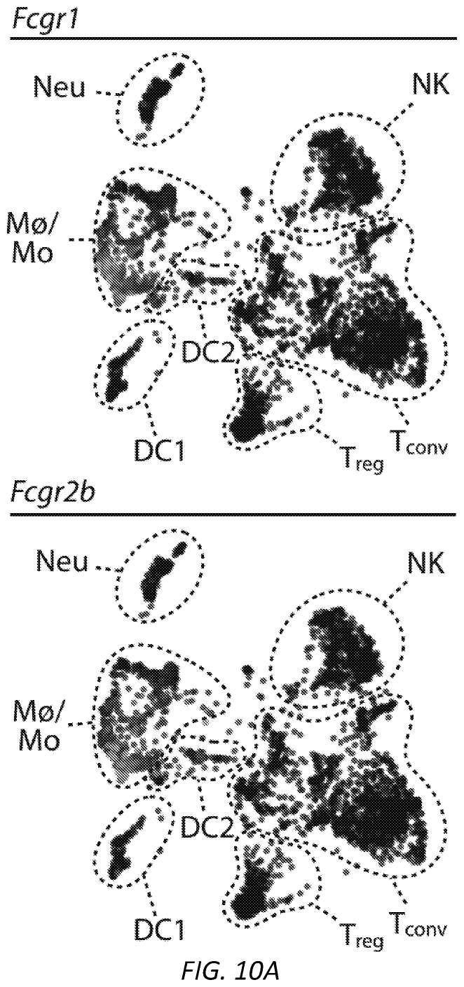

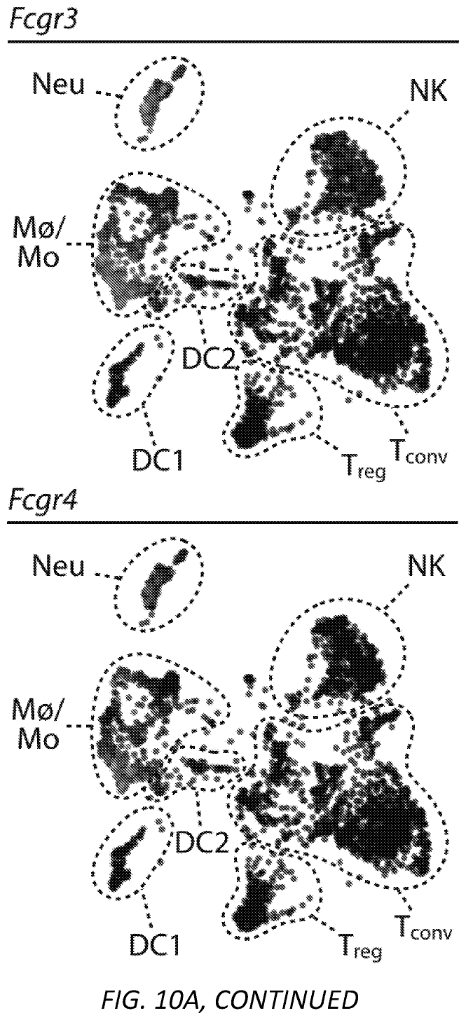

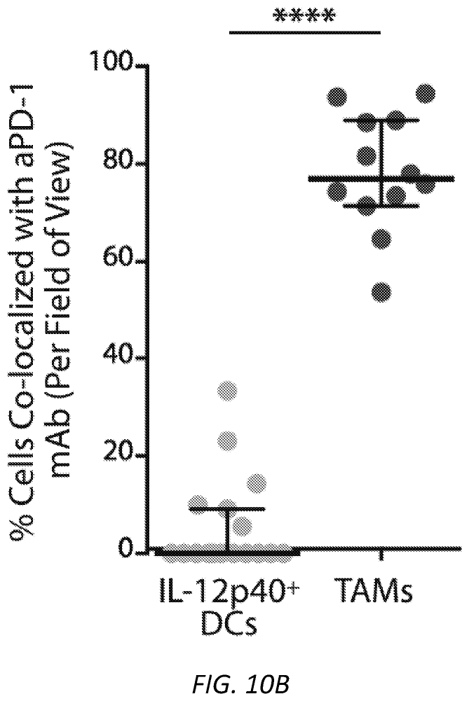

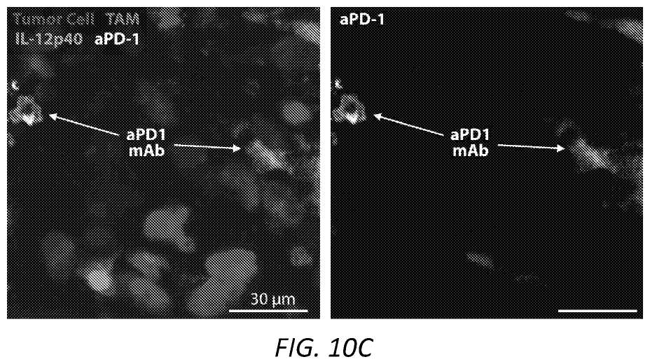

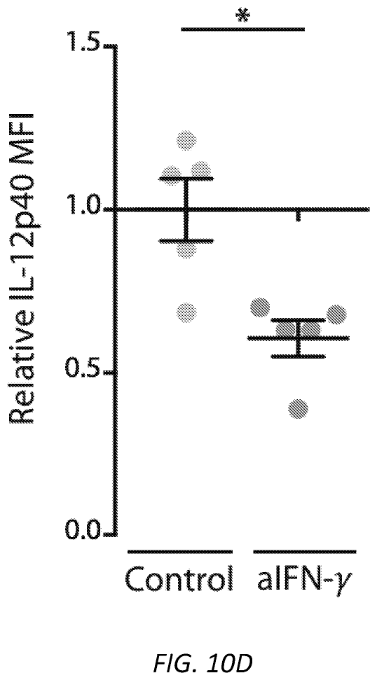

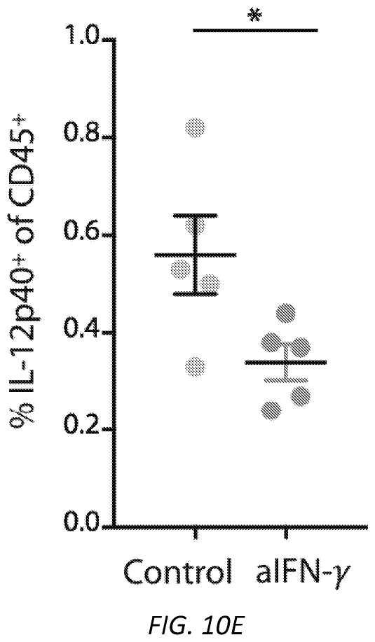

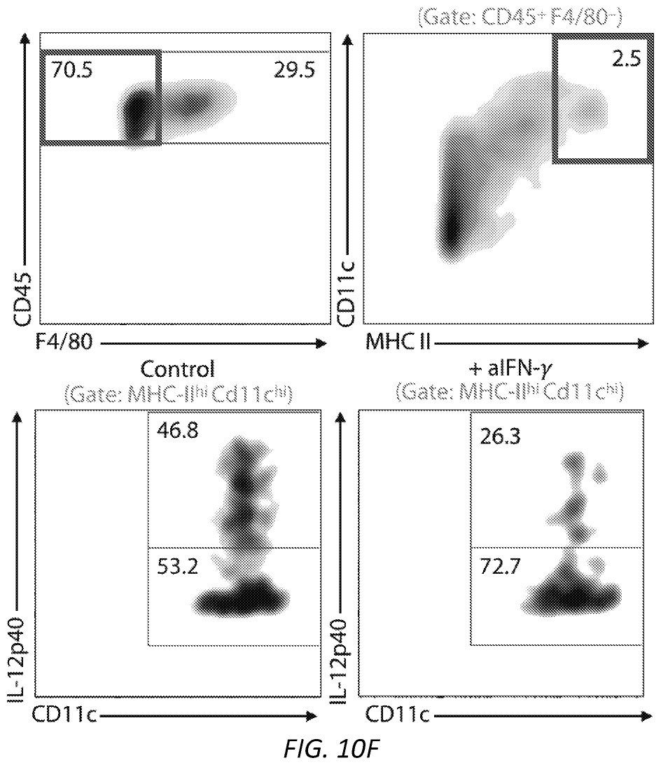

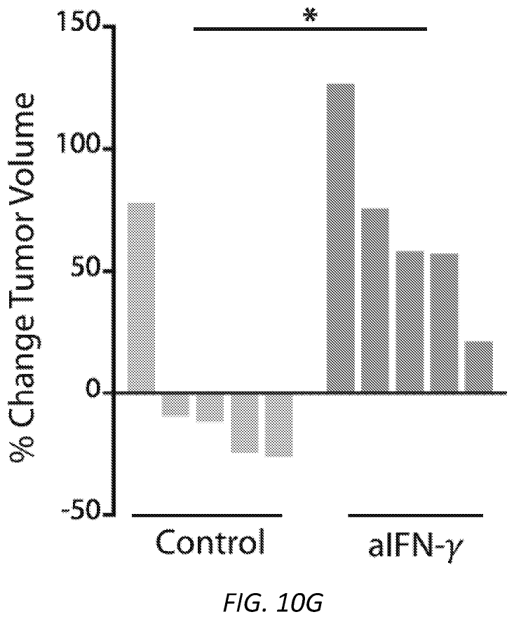

To define how aPD-1 treatment activates DCs, we asked initially whether the antibody binds to these cells directly. Some myeloid cells have been proposed to express PD-1 (Gordon et al., 2017); however, both flow cytometry and scRNAseq analyses indicated that IL-12⁺ DCs did not express the PD-1 receptor at both transcript (FIG. 9A) and protein (FIG. 3A) levels. We further tested whether aPD-1 antibodies bind IL-12⁺ DCs independently of PD-1. Indeed, aPD-1 mAbs initially accumulate on PD-1⁺ T cells but can then be gradually taken up by tumor-associated macrophages (TAMs) in a FcγR-dependent manner (Arlauckas et al., 2017). However, IL-12⁺ DCs did not express detectable levels of FcγR transcripts, in contrast to TAMs (FIG. 10A). Also, when tracking the drug's pharmacokinetics by intravital imaging in MC38 tumor-bearing IL-12-reporter mice, we confirmed aPD-1 accumulation in TAMs but not in IL-12⁺ DCs 24 hours after aPD-1 administration (FIG. 3B and FIG. 10B). The DCs also failed to bind aPD-1 early after drug administration, i.e. before uptake by TAMs (FIG. 10C). Based on these data we concluded that it was unlikely for aPD-1 to bind and activate IL-12⁺ DCs directly.

As aPD-1 mAbs physically bind to tumor-infiltrating CD8⁺ T cells (Arlauckas et al., 2017), we hypothesized that these cells, once activated by aPD-1, could promote IL-12 production by DCs. To address this possibility, we used intravital imaging to track IL-12 expression in mice depleted of CD8⁺ T cells prior to administration of aPD-1. Absence of CD8⁺ T cells abrogated IL-12 production (FIG. 3C). We further reasoned that IFN-γ could mediate IL-12 production by DCs, since this cytokine was produced by aPD-1-activated CD8⁺ T cells (FIG. 1B) and can enhance IL-12 responses (Ma et al., 1996). To test this hypothesis, we assessed mice in which IFN-γ was neutralized during aPD-1 treatment. We found that IFN-γ blockade reduced IL-12 production within the tumor microenvironment (FIG. 3D). Decreased IL-12 production by DCs (FIG. 10D) and decreased numbers of IL-12⁺ DCs (FIG. 10E, F) both contributed to this reduction. Consequently, IFN-γ blockade prevented aPD-1-mediated MC38 tumor control (FIG. 10G).

The above results suggest that IFN-γ sensing by DCs fosters IL-12 production and results in tumor control. To test this hypothesis directly, we eliminated DC sensing of IFN-γ by crossing Itgax-cre with Ifngr 1^{fl/fl} mice (Lee et al., 2013). Tumors from these mice showed impaired IL-12p40 production (FIG. 3E) and were unresponsive to aPD-1 treatment (FIG. 3F), underscoring the importance of IFN-γ sensing by DCs, and potentially other CD11c expressing cells, during aPD-1 therapy. Prior studies of Ifngr1-deficient DCs (Nirschl et al., 2017) described down-regulation of genes such as Fscn1, Ccr7, and Icam1, which we identified as IL-12⁺ DC distinguishers by scRNAseq analysis (FIG. 2B, D, E). Together, we find an indirect aPD-1 effect on DCs; this effect was mediated through IFN-γ and is critical for IL-12 induction and, consequently, treatment response.

### Example 5. IL-12 Activates Tumor-Infiltrating Lymphocyte Effector Functions in Mice

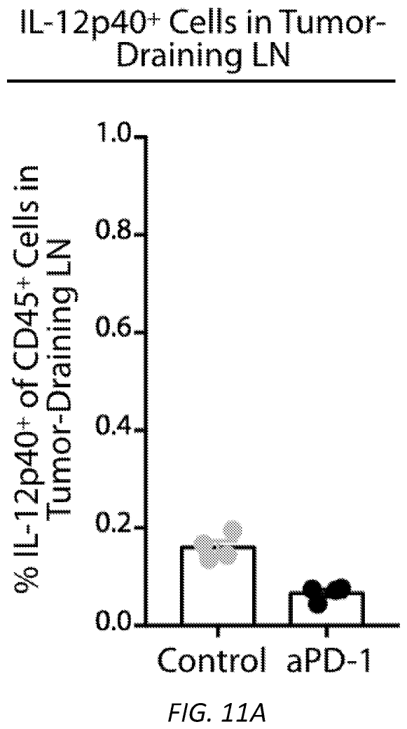

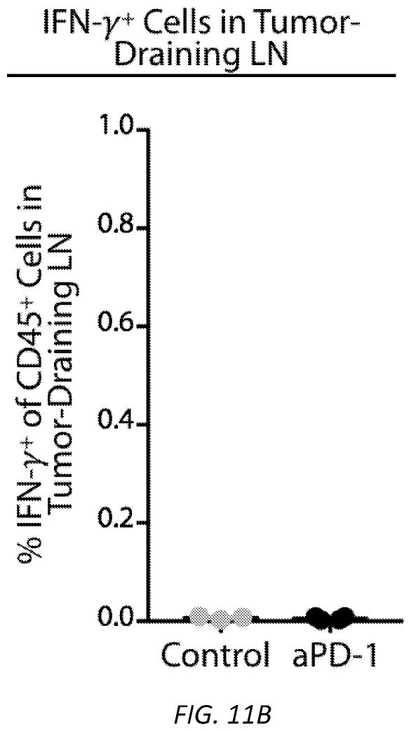

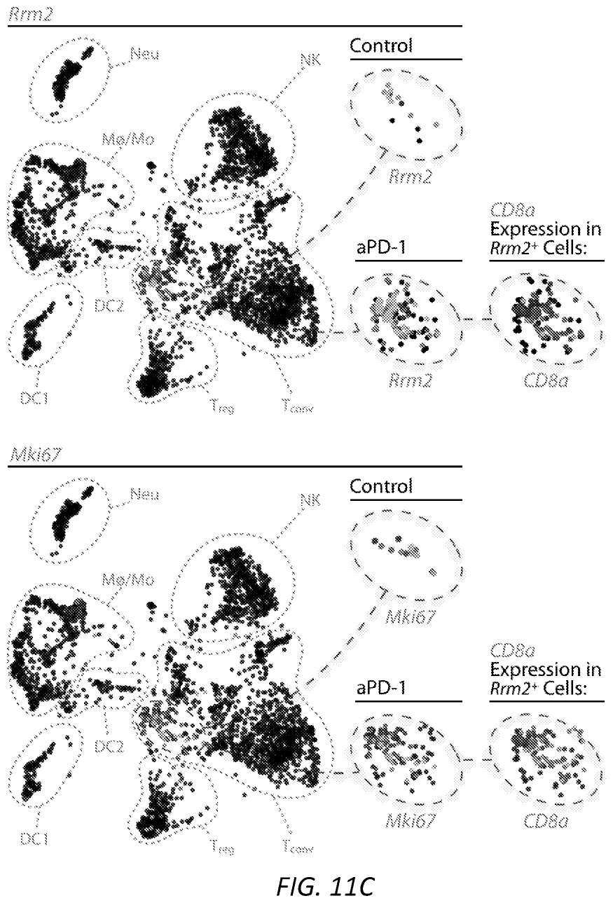

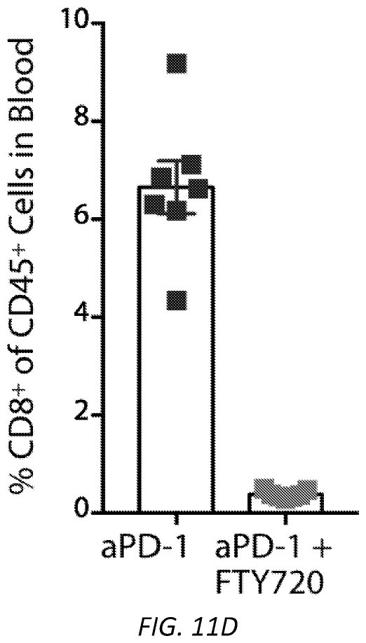

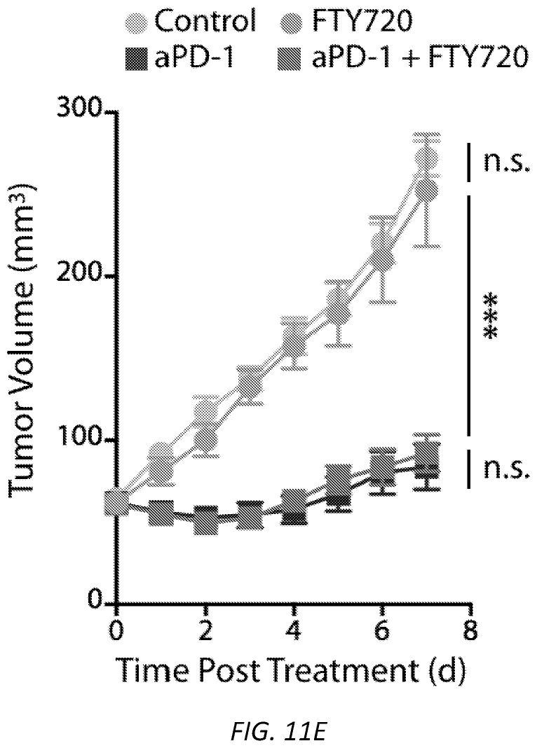

Our investigations indicated that aPD-1 treatment elicits both IFN-γ and IL-12 responses at the tumor site. By contrast, we did not find evidence of IFN-γ or IL-12 induction by aPD-1 in the local draining lymph node (FIGS. 11A and B), suggesting that the checkpoint blockade response occurs within tumors. Consistent with this notion, scRNAseq data indicated that aPD-1 treatment triggered the proliferation of tumor-infiltrating CD8⁺ T cells (FIG. 11C). Furthermore, blocking lymphocyte recirculation through treatment with the trafficking inhibitor FTY720 did not affect the antitumor response to aPD-1 treatment (FIGS. 11D and E). These data suggest that pre-existing tumor-infiltrating T cells are sufficient for driving the response to aPD-1 at least in this model.

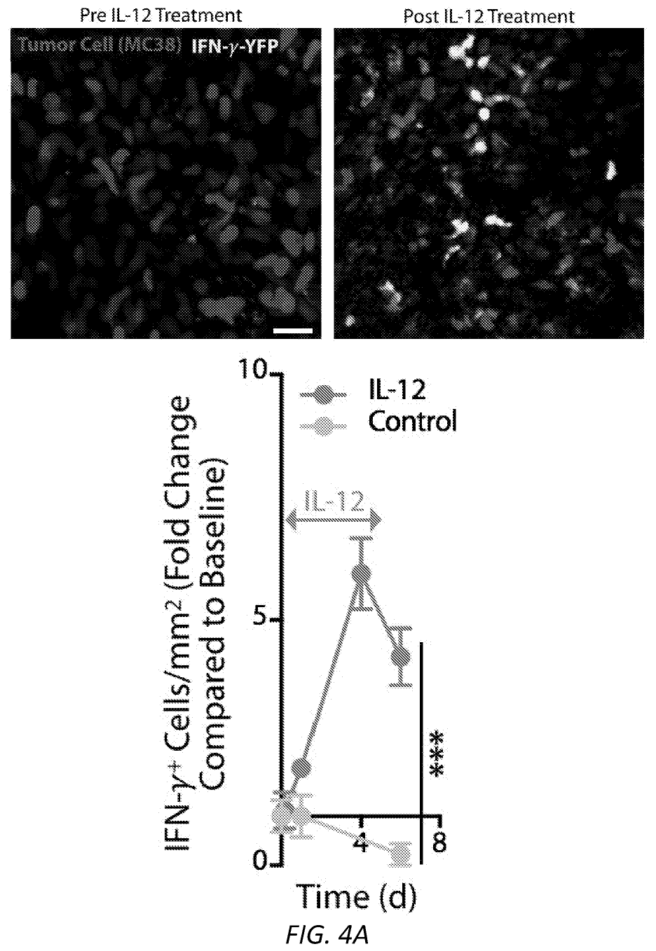

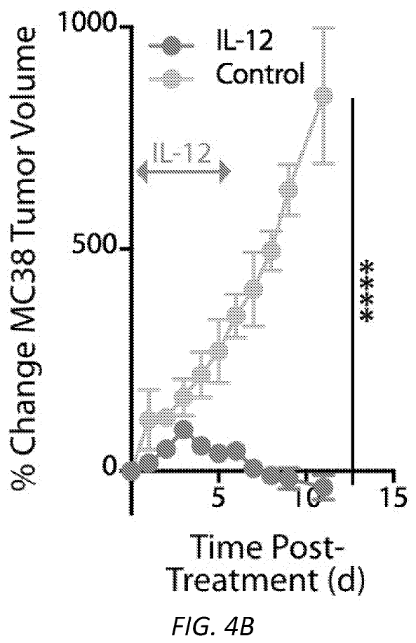

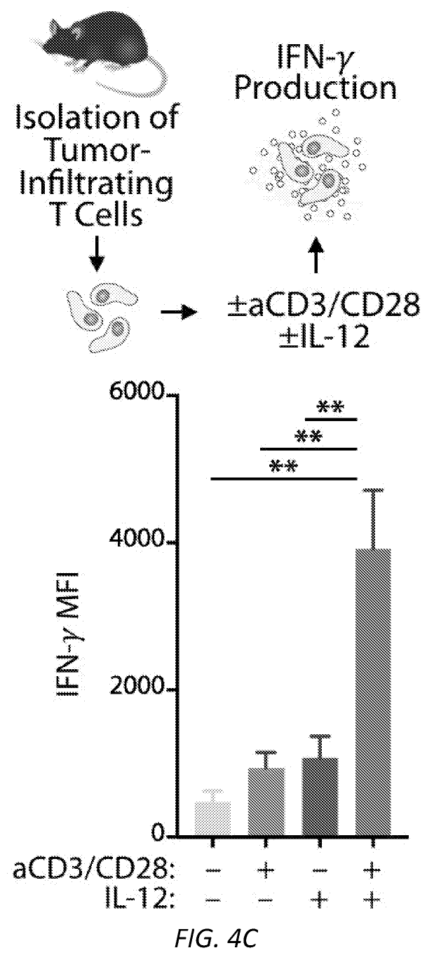

We next examined the downstream effects of IL-12 production within the tumor microenvironment. Initially we used intravital microscopy to assess the effects of recombinant IL-12 administered to tumors in IFN-γ reporter mice (in the absence of aPD-1). We found that intratumoral IL-12 substantially expanded IFN-γ-eYFP⁺ cells (5.9±0.7 fold increase by day four; FIG. 4A). Consistent with previous reports (Nastala et al., 1994), IL-12 administration to MC38 tumors produced robust antitumor responses (FIG. 4B). To test further whether IL-12 can activate tumor-infiltrated CD8⁺ T cells directly, we isolated these cells from MC38 tumors and subjected them to aCD3/CD28 stimulation with or without IL-12. Stimulated CD8⁺ T cells substantially increased IFN-γ production in the presence of IL-12 (FIG. 4C), indicating that tumor-infiltrating T cells can respond to IL-12 directly. The requirement for both T cell co-stimulation and IL-12 to achieve maximal IFN-γ response likely reflected the need of CD28 to rescue exhausted CD8⁺ T cells, and possibly also the role of PD-1 in limiting CD28-mediated co-stimulation (Kamphorst et al., 2017; Hui et al., 2017).

### Example 6. IL-12 Activates Tumor-Infiltrating Lymphocyte Effector Functions in Cancer Patients

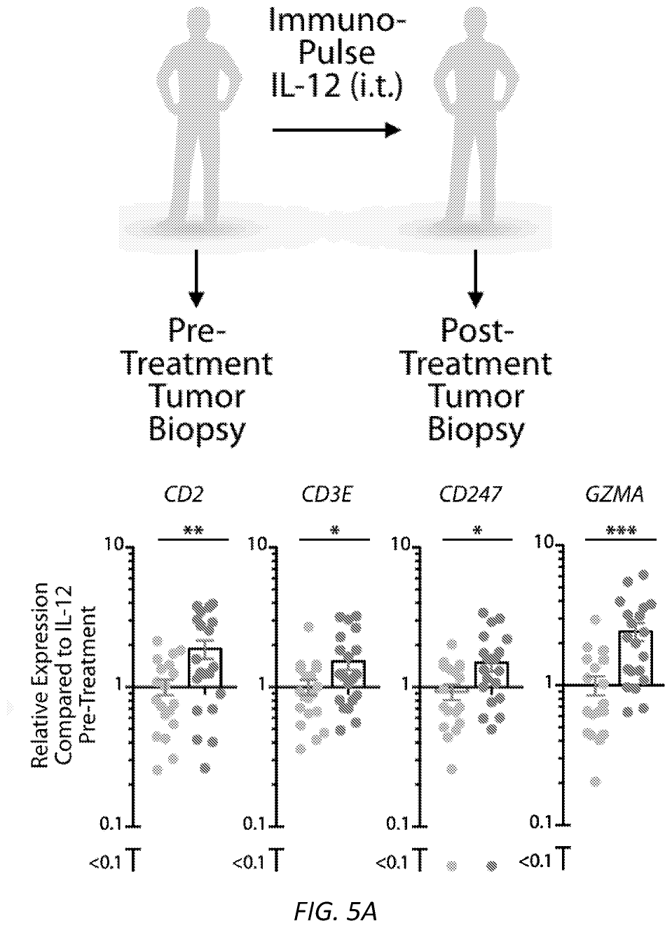

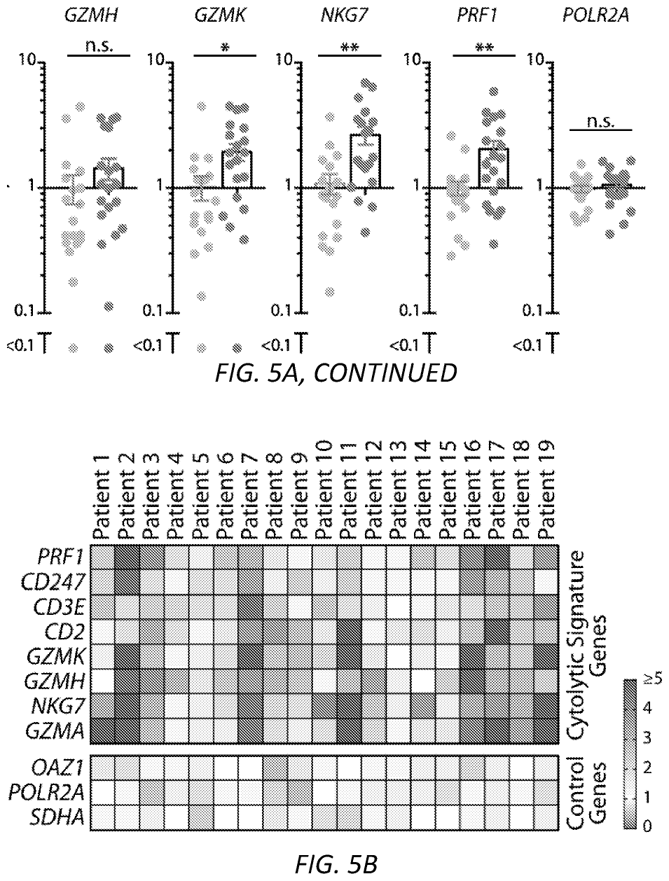

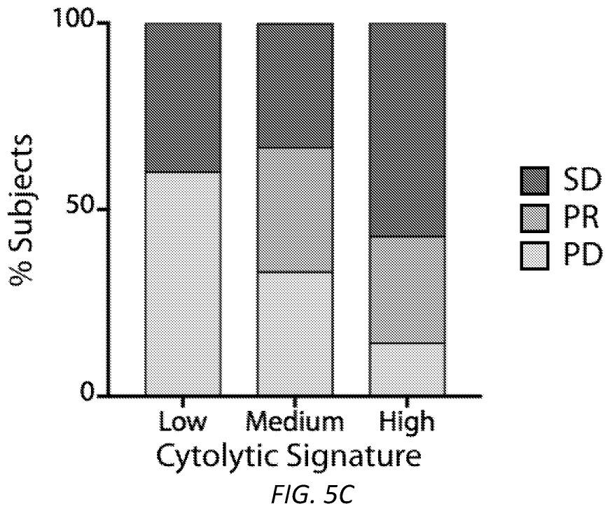

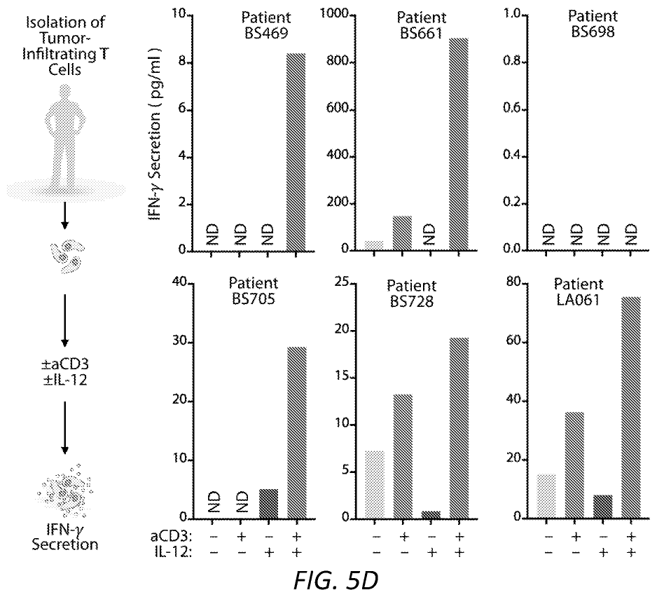

We next addressed the downstream effects of IL-12 in cancer patients using two clinical cohorts. First, to assess IL-12's effects within tumors, we collected skin tumor biopsies from 19 melanoma patients both before and after intratumoral treatment with ImmunoPulse tavokinogene telseplasmid, an electroporation method that delivers plasmid IL-12 directly to tumors (Daud et al., 2008). Comparison of pre- and post-treatment samples revealed that IL-12 delivery enhanced expression of core cytolytic genes (Rooney et al., 2015) within tumors (FIG. 5A, B). These genes, namely CD2, CD3E, CD274, GZMA, GZMH, GZMK, NKG7 and PRF1, are associated with immunoediting and antitumor immune responses (Rooney et al., 2015) and tumors enriched with these genes are more likely to respond to aPD-1 immunotherapy (Riaz et al., 2017). Accordingly, we observed a positive association between enhanced cytolytic gene signature and therapeutic response in these patients (FIG. 5C). IFNG was not detectably increased in the post-treatment samples, which is expected from the timing of tissue collection and rapid on/off cycling of IFN-γ production by T cells (Slifka et al., 1999). These observations indicated that IL-12 can induce cytolytic activity in human tumors.

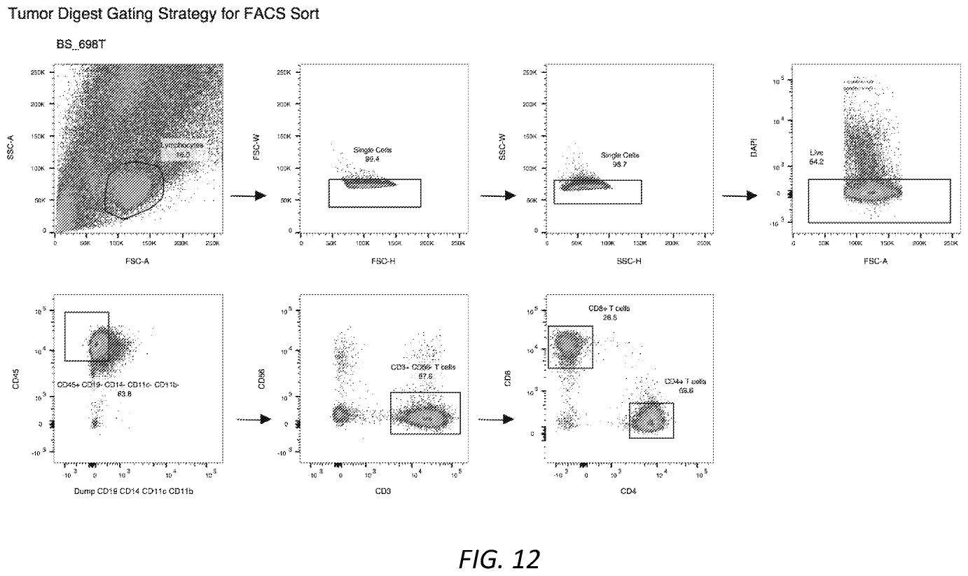

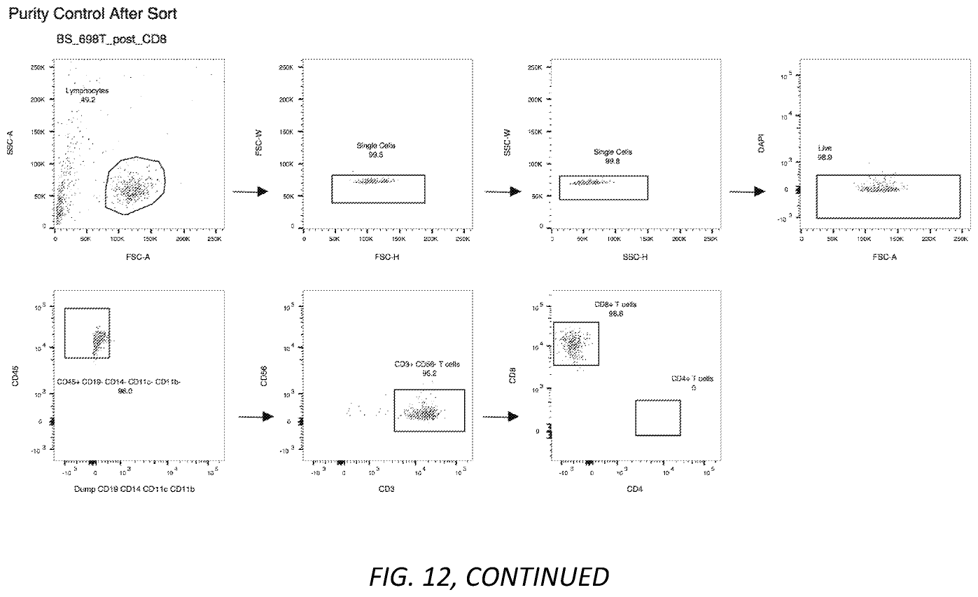

To define whether IL-12 can directly activate human tumor-infiltrating CD8⁺ T cells upon isolation of these cells, we collected fresh tumor tissue from six cancer patients, which included two lung adenocarcinomas (patients BS728 and LA061), three lung squamous cell carcinomas (patients BS469, BS698 and BS705) and one synovial sarcoma (patient BS661). CD8⁺ T cells were purified from all tumors ex vivo (FIG. 12) and subjected to aCD3 stimulation with or without IL-12. The presence of IL-12 increased IFN-γ production by CD8⁺ T cells in five out of six patients (FIG. 5D). Collectively, these patient data recapitulate our observations in mice that IL-12 can directly stimulate tumor-infiltrating T cell antitumor activity. They also support previous evidence that CD8⁺ T cell activation within tumors is critical to antitumor activity (Broz et al., 2014; Spranger et al., 2014).

### Example 7. Activation of the Non-Canonical NFkB Pathway Amplifies IL-12-Producing DCs

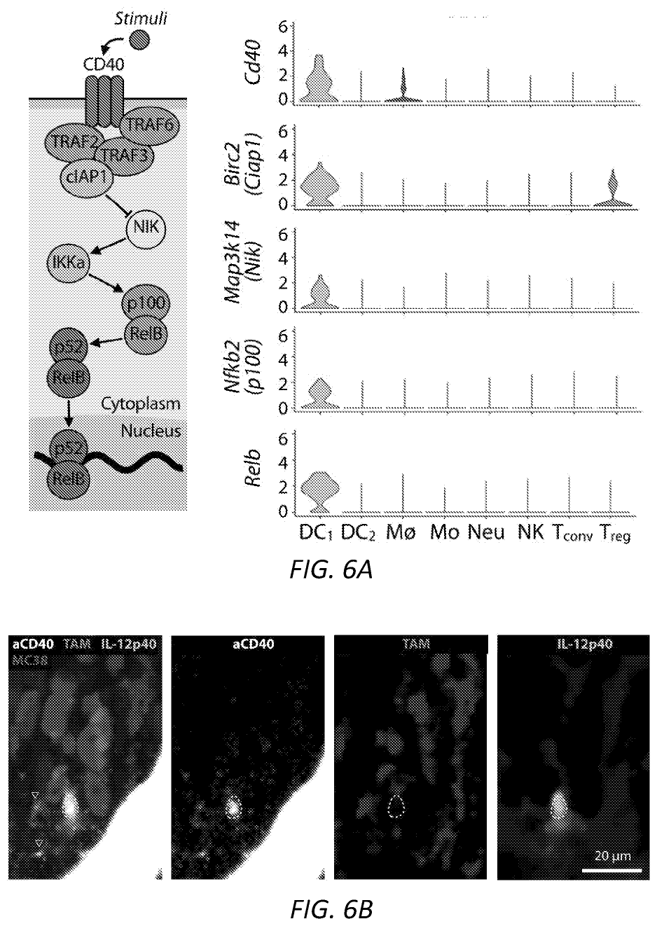

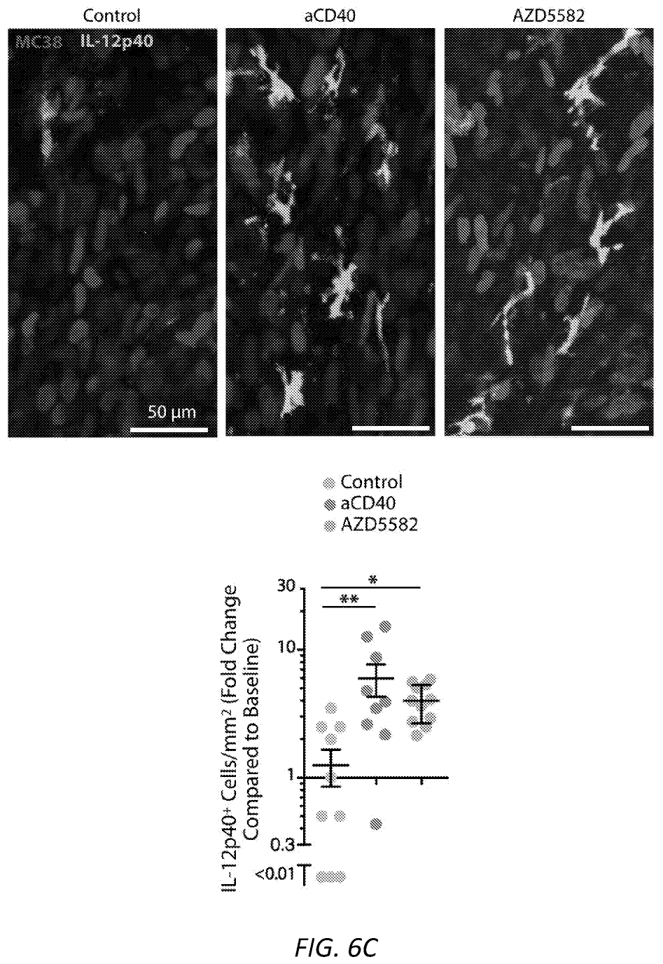

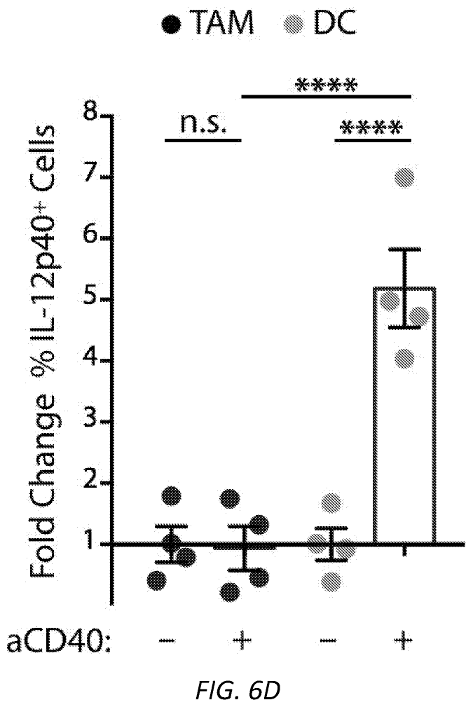

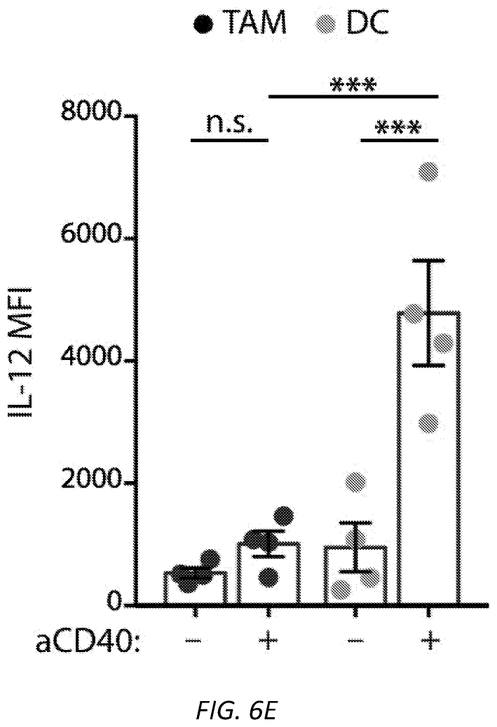

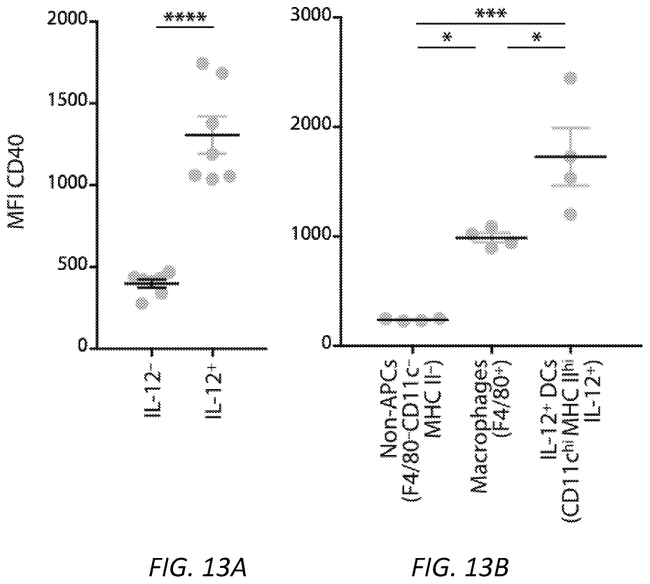

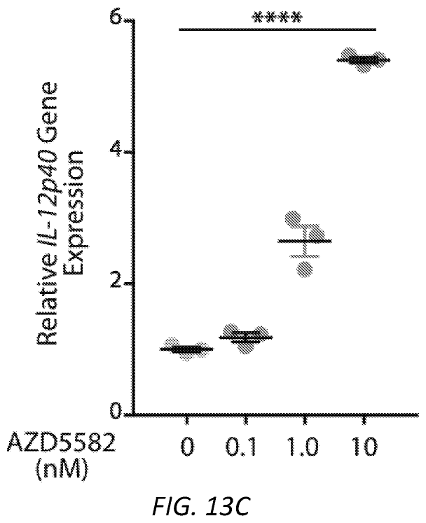

On account of IL-12's ability to license antitumor T cell immunity, we further asked whether agonizing IL-12-producing cells could augment response to aPD-1 therapy. We examined the non-canonical NFkB pathway as a therapeutic target, considering its relevance for priming cytotoxic T cells (Katakam et al., 2015; Lind et al., 2008) and because key non-canonical NFkB pathway genes, namely Cd40, Birc2 (Ciap1), Map3k14 (Nik), Nfkb2 (p100) and Relb, were all selectively up-regulated in the IL-12⁺ tumor-infiltrating DC subset (FIG. 6A). We confirmed that IL-12⁺ cells had more cell surface CD40 than their IL-12⁻ counterparts (FIG. 13A) and that IL-12⁺ DCs expressed more CD40 than tumor-associated macrophages (FIG. 13B).

We sought to activate the non-canonical NFkB pathway in two different ways: with agonistic CD40 mAbs that have previously shown antitumor activity (Beatty et al., 2011; Byrne and Vonderheide, 2016) or with the small molecule inhibitor AZD5582 that targets cellular inhibitor of apoptosis protein (cIAP) 1 and 2 (Hennessy et al., 2013). Agonistic aCD40 mAbs were labeled with a fluorescent dye and tracked by intravital microscopy within tumors of IL-12 reporter mice. This imaging approach not only showed the drug's ability to interact directly with IL-12⁺ tumor-infiltrating cells, and some macrophages, in vivo (FIG. 6B) but further identified that the treatment induced a 6.6±1.2-fold increase of tumor-infiltrating IL-12⁺ cells (FIG. 6C). Flow cytometry measurements indicated that IL-12 was produced by DCs but not TAMs (FIG. 6D, E). These findings align with previous evidence that aCD40 therapy relies upon Batf3-dependent DCs (Byrne and Vonderheide, 2016), although macrophages can also contribute to aCD40 therapy in some settings, which may be independent of IL-12 (Hoves et al., 2018; Beatty et al., 2011).

CD40, in addition to activating myeloid cells, is also a well-known activator of B cells. Therefore, we tested if B cells were important for aCD40 therapy response. We found that B cell depletion had no effect on aCD40 therapy, suggesting that B cells are not necessary for aCD40 treatment in this experimental model (data not shown).

Treating tumors with the cIAP antagonist AZD5582 induced a 4.0±1.3-fold increase of IL-12⁺ tumor-infiltrating cells (FIG. 6C), similar to the effects observed with agonistic CD40 mAbs. Furthermore, stimulation of Flt3L-derived bone marrow DCs with AZD5582 potently enhanced IL-12 production in vitro (FIG. 13C). These results not only confirm previous evidence that CD40 agonism is a stimulatory signal for DCs (Cella et al., 1996; Ngiow et al., 2016) but also indicate that triggering the non-canonical NFkB pathway, through CD40 agonism or cIAP inhibition, can amplify IL-12⁺ tumor-infiltrating DCs.

### Example 8. Amplification of IL-12⁺ DCs Improves Cancer Immunotherapy in an IL-12-Dependent Manner

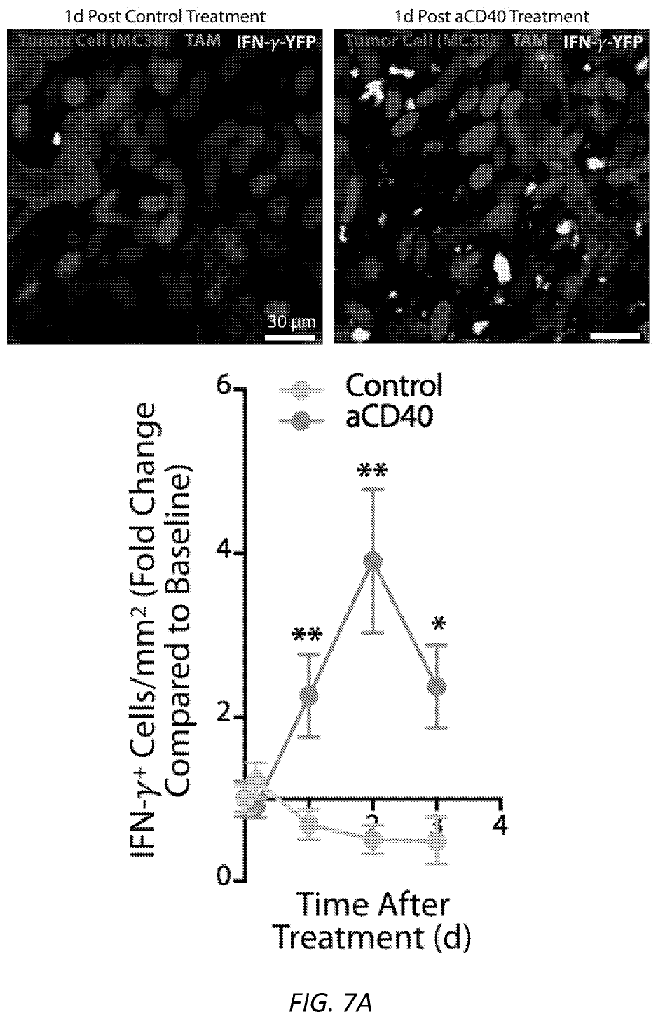

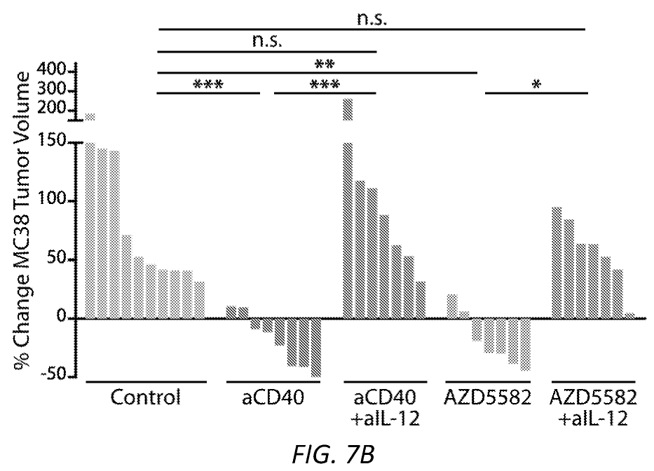

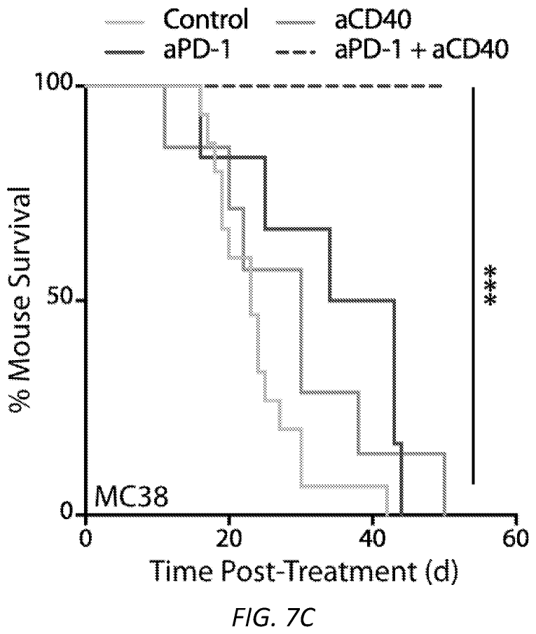

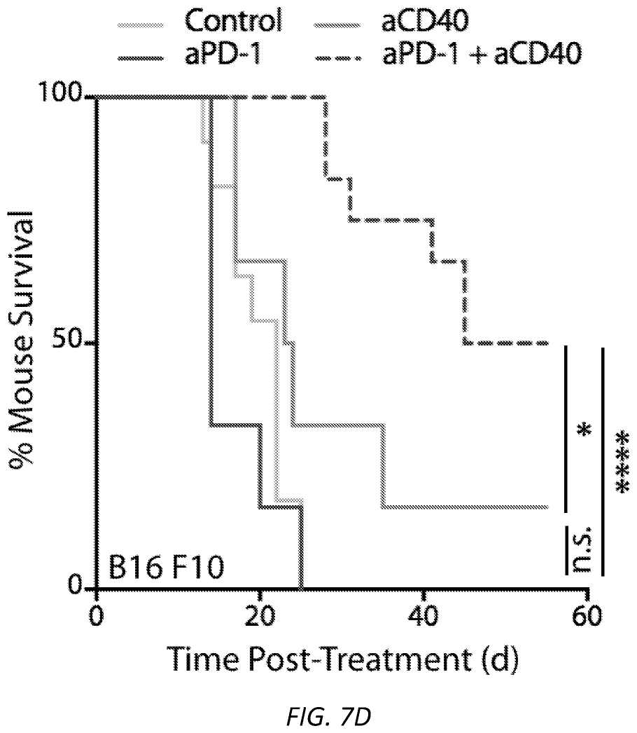

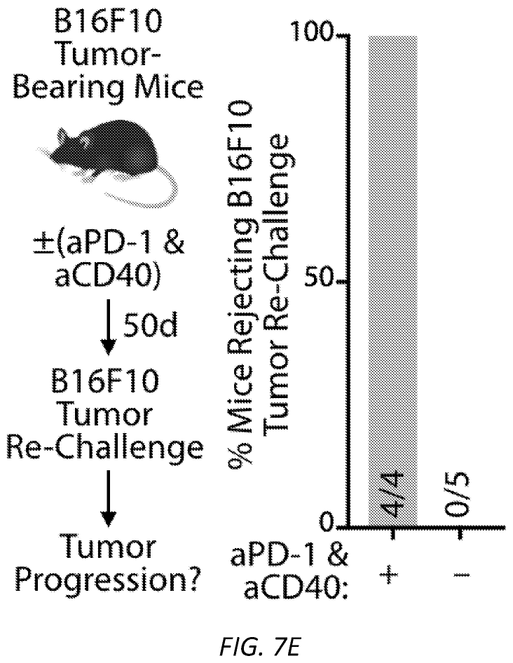

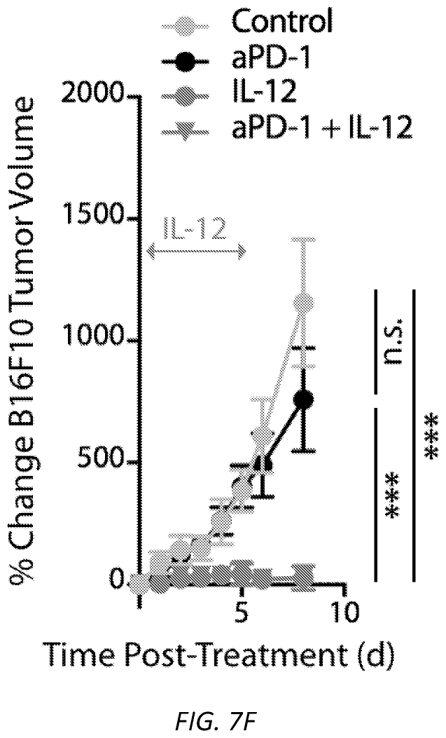

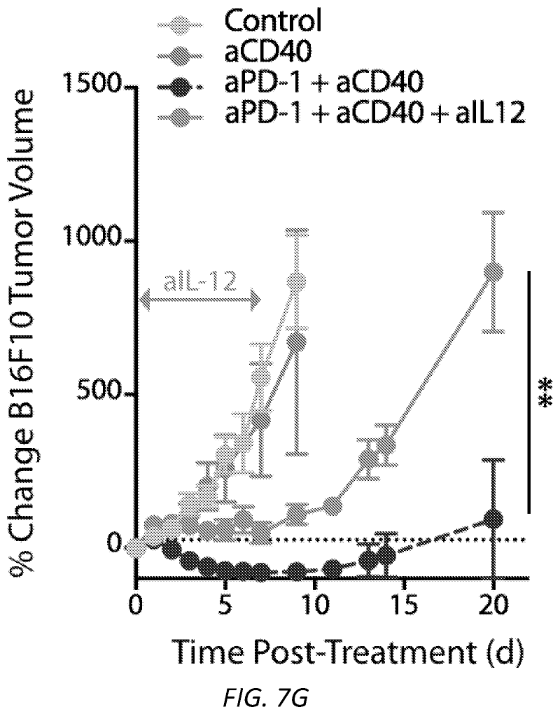

The antitumor activity of agonistic CD40 mAbs (aCD40) has been shown to depend upon IFN-γ (Byrne and Vonderheide, 2016). We evaluated aCD40 in IFN-γ reporter animals and indeed found that aCD40 treatment potently enhanced intratumoral IFN-γ levels (FIG. 7A). The IFN-γ induction by aCD40 likely occurred indirectly as T cells did not express CD40 (FIG. 6A). Furthermore, treatment with either agonistic aCD40 mAbs or AZD5582 provided antitumor effects in vivo (FIG. 7B). To test the relevance of IL-12 following treatments with aCD40 or AZD5582, we compared their effects in MC38 tumor-bearing mice that were administered or not with IL-12 neutralizing mAbs. These studies showed that IL-12 induction was a primary mechanism for these treatments because tumor control was lost in animals receiving IL-12 neutralizing mAbs (FIG. 7B). To further assess the requirement of non-canonical NFkB signaling to aPD-1 treatment efficacy, we compared aPD-1 responses in mice that were reconstituted with either Map3k14 (NIK) deficient or wild-type bone marrow. NIK chimeras failed to respond to aPD-1 (FIG. 14A). Taken together, these data linked the non-canonical NFkB pathway to antitumor intratumoral DCs and to aPD-1 treatment efficacy, and indicated that targeting non-canonical NFkB components can be therapeutic in cancer.

Next we defined whether agonizing IL-12⁺ cells could augment response to aPD-1 therapy. To this end, we assessed MC38 tumor progression in mice treated with antagonist aPD-1, agonist aCD40 or both. We found that monotherapies incompletely controlled tumor growth, whereas the combination treatment produced a complete, durable response in most animals treated (FIG. 7C and FIGS. 14B and C). Mice that received combination treatment further resisted tumor re-challenge 8 weeks after the primary tumor rejection (FIG. 14D); this indicated that the treatment had triggered antitumor memory.

Because the MC38 tumor model responds—though not completely—to aPD-1 monotherapy, we also tested the B16F10 melanoma model, which resists aPD-1 treatment. We found that combining aPD-1 with aCD40 mAbs controlled B16F10 tumor growth (FIGS. 14E and F) and resulted in increased mouse survival (FIG. 7D), when compared to aPD-1 or aCD40 monotherapies. The combination treatment rejected tumors in 50% (6 of 12) mice; these mice resisted secondary tumor challenge (FIG. 7E), indicating that the treatment had also triggered antitumor memory in this model.

Considering that recombinant IL-12 administered to B16F10 melanoma-bearing mice also produced a substantial antitumor effect (FIG. 7F), we tested whether the aPD-1+aCD40 therapeutic combination relied upon IL-12 for activity. We administered the combination immunotherapy to B16F10-bearing mice in the presence or absence of IL-12 neutralizing mAbs, and found that blocking IL-12 signaling prevented the combination treatment's therapeutic activity (FIG. 7G and FIG. 14G). These data indicate that DC targeting can augment immunotherapy efficacy and sensitize tumors to aPD-1 treatment in an IL-12-dependent manner.

### REFERENCES

Alloatti, A., Rookhuizen, D. C., Joannas, L., Carpier, J. M., Iborra, S., Magalhaes, J. G., Yatim, N., Kozik, P., Sancho, D., Albert, M. L. et al. (2017). Critical role for Sec22b-dependent antigen cross-presentation in antitumor immunity. J Exp Med 214, 2231-2241.
Arlauckas, S. P., Garris, C. S., Kohler, R. H., Kitaoka, M., Cuccarese, M. F., Yang, K. S., Miller, M. A., Carlson, J. C., Freeman, G. J., Anthony, R. M. et al. (2017). In vivo imaging reveals a tumor-associated macrophage-mediated resistance pathway in anti-PD-1 therapy. Sci Transl Med 9, eaa13604.
Ayers, M., Lunceford, J., Nebozhyn, M., Murphy, E., Loboda, A., Kaufman, D. R., Albright, A., Cheng, J. D., Kang, S. P., Shankaran, V. et al. (2017). IFN-γ-related mRNA profile predicts clinical response to PD-1 blockade. J Clin Invest 127, 2930-2940.
Beatty, G. L., Chiorean, E. G., Fishman, M. P., Saboury, B., Teitelbaum, U. R., Sun, W., Huhn, R. D., Song, W., Li, D., Sharp, L. L. et al. (2011). CD40 agonists alter tumor stroma and show efficacy against pancreatic carcinoma in mice and humans. Science 331, 1612-1616.
Broz, M. L., Binnewies, M., Boldajipour, B., Nelson, A. E., Pollack, J. L., Erle, D. J., Barczak, A., Rosenblum, M. D., Daud, A., Barber, D. L. et al. (2014). Dissecting the tumor myeloid compartment reveals rare activating antigen-presenting cells critical for T cell immunity. Cancer Cell 26, 638-652.
Byrne, K. T., and Vonderheide, R. H. (2016). CD40 stimulation obviates innate sensors and drives T cell immunity in cancer. Cell reports 15, 2719-2732.
Cella, M., Scheidegger, D., Palmer-Lehmann, K., Lane, P., Lanzavecchia, A., and Alber, G. (1996). Ligation of CD40 on dendritic cells triggers production of high levels of interleukin-12 and enhances T cell stimulatory capacity: T-T help via APC activation. J Exp Med 184, 747-752.
Dahan, R., Sega, E., Engelhardt, J., Selby, M., Korman, A. J., and Ravetch, J. V. (2015). FcγRs Modulate the Anti-tumor Activity of Antibodies Targeting the PD-1/PD-L1 Axis. Cancer Cell 28, 285-295.
Daud, A. I., DeConti, R. C., Andrews, S., Urbas, P., Riker, A. I., Sondak, V. K., Munster, P. N., Sullivan, D. M., Ugen, K. E., Messina, J. L. et al. (2008). Phase I trial of interleukin-12 plasmid electroporation in patients with metastatic melanoma. J Clin Oncol 26, 5896-5903.
de Mingo Pulido, Á., Gardner, A., Hiebler, S., Soliman, H., Rugo, H. S., Krummel, M. F., Coussens, L. M., and Ruffell, B. (2018). TIM-3 Regulates CD103+ Dendritic Cell Function and Response to Chemotherapy in Breast Cancer. Cancer cell 33, 60-74. e6.
Engblom, C., Pfirschke, C., and Pittet, M. J. (2016). The role of myeloid cells in cancer therapies. Nat Rev Cancer 16, 447-462.
Galon, J., Angell, H. K., Bedognetti, D., and Marincola, F. M. (2013). The continuum of cancer immunosurveillance: prognostic, predictive, and mechanistic signatures. Immunity 39, 11-26.
Gordon, S. R., Maute, R. L., Dulken, B. W., Hutter, G., George, B. M., McCracken, M. N., Gupta, R., Tsai, J. M., Sinha, R., Corey, D. et al. (2017). PD-1 expression by tumour-associated macrophages inhibits phagocytosis and tumour immunity. Nature 545, 495-499.
Hennessy, E. J., Adam, A., Aquila, B. M., Castriotta, L. M., Cook, D., Hattersley, M., Hird, A. W., Huntington, C., Kamhi, V. M., Laing, N. M. et al. (2013). Discovery of a novel class of dimeric Smac mimetics as potent IAP antagonists resulting in a clinical candidate for the treatment of cancer (AZD5582). J Med Chem 56, 9897-9919.
Hildner, K., Edelson, B. T., Purtha, W. E., Diamond, M., Matsushita, H., Kohyama, M., Calderon, B., Schraml, B. U., Unanue, E. R., Diamond, M. S. et al. (2008). Batf3 deficiency reveals a critical role for CD8alpha+ dendritic cells in cytotoxic T cell immunity. Science 322, 1097-1100.
Hoves, S., Ooi, C.-H., Wolter, C., Sade, H., Bissinger, S., Schmittnaegel, M., Ast, O., Giusti, A. M., Wartha, K., and Runza, V. (2018). Rapid activation of tumor-associated macrophages boosts preexisting tumor immunity. Journal of Experimental Medicine jem. 20171440.
Huang, A. C., Postow, M. A., Orlowski, R. J., Mick, R., Bengsch, B., Manne, S., Xu, W., Harmon, S., Giles, J. R., Wenz, B. et al. (2017). T-cell invigoration to tumour burden ratio associated with anti-PD-1 response. Nature 545, 60-65.
Hui, E., Cheung, J., Zhu, J., Su, X., Taylor, M. J., Wallweber, H. A., Sasmal, D. K., Huang, J., Kim, J. M., Mellman, I. et al. (2017). T cell costimulatory receptor CD28 is a primary target for PD-1-mediated inhibition. Science 355, 1428-1433.
Joshi, N. S., Cui, W., Chandele, A., Lee, H. K., Urso, D. R., Hagman, J., Gapin, L., and Kaech, S. M. (2007). Inflammation directs memory precursor and short-lived effector CD8(+) T cell fates via the graded expression of T-bet transcription factor. Immunity 27, 281-295.
Kamphorst, A. O., Wieland, A., Nasti, T., Yang, S., Zhang, R., Barber, D. L., Konieczny, B. T., Daugherty, C. Z., Koenig, L., and Yu, K. (2017). Rescue of exhausted CD8 T cells by PD-1-targeted therapies is CD28-dependent. Science 355, 1423-1427.
Kao, C., Oestreich, K. J., Paley, M. A., Crawford, A., Angelosanto, J. M., Ali, M. A., Intlekofer, A. M., Boss, J. M., Reiner, S. L., Weinmann, A. S. et al. (2011). Transcription factor T-bet represses expression of the inhibitory receptor PD-1 and sustains virus-specific CD8+ T cell responses during chronic infection. Nat Immunol 12, 663-671.
Katakam, A. K., Brightbill, H., Franci, C., Kung, C., Nunez, V., Jones, C., Peng, I., Jeet, S., Wu, L. C., and Mellman, I. (2015). Dendritic cells require NIK for CD40-dependent cross-priming of CD8+ T cells. Proceedings of the National Academy of Sciences 112, 14664-14669.
Keir, M. E., Butte, M. J., Freeman, G. J., and Sharpe, A. H. (2008). PD-1 and its ligands in tolerance and immunity. Annu. Rev. Immunol. 26, 677-704.
Lasek, W., Zagożdżon, R., and Jakobisiak, M. (2014). Interleukin 12: still a promising candidate for tumor immunotherapy. Cancer Immunol Immunother 63, 419-435.
Lee, S. H., Carrero, J. A., Uppaluri, R., White, J. M., Archambault, J. M., Lai, K. S., Chan, S. R., Sheehan, K. C., Unanue, E. R., and Schreiber, R. D. (2013). Identifying the initiating events of anti-Listeria responses using mice with conditional loss of IFN-γ receptor subunit 1 (IFNGR1). J Immunol 191, 4223-4234.
Lind, E. F., Ahonen, C. L., Wasiuk, A., Kosaka, Y., Becher, B., Bennett, K. A., and Noelle, R. J. (2008). Dendritic cells require the NF-κB2 pathway for cross-presentation of soluble antigens. The Journal of Immunology 181, 354-363.
Ma, X., Chow, J. M., Gri, G., Carra, G., Gerosa, F., Wolf, S. F., Dzialo, R., and Trinchieri, G. (1996). The interleukin 12 p40 gene promoter is primed by interferon gamma in monocytic cells. J Exp Med 183, 147-157.
Macosko, E. Z., Basu, A., Satija, R., Nemesh, J., Shekhar, K., Goldman, M., Tirosh, I., Bialas, A. R., Kamitaki, N., Martersteck, E. M. et al. (2015). Highly Parallel Genome-wide Expression Profiling of Individual Cells Using Nanoliter Droplets. Cell 161, 1202-1214.
Martin-Fontecha, A., Sebastiani, S., Hopken, U. E., Uguccioni, M., Lipp, M., Lanzavecchia, A., and Sallusto, F. (2003). Regulation of dendritic cell migration to the draining lymph node: impact on T lymphocyte traffic and priming. J Exp Med 198, 615-621.
Marty, R., Kaabinejadian, S., Rossell, D., Slifker, M. J., van de Haar, J., Engin, H. B., de Prisco, N., Ideker, T., Hildebrand, W. H., Font-Burgada, J. et al. (2017). MHC-I Genotype Restricts the Oncogenic Mutational Landscape. Cell 171, 1272-1283.e15.
Matson, V., Fessler, J., Bao, R., Chongsuwat, T., Zha, Y., Alegre, M. L., Luke, J. J., and Gajewski, T. F. (2018). The commensal microbiome is associated with anti-PD-1 efficacy in metastatic melanoma patients. Science 359, 104-108.
McGranahan, N., Rosenthal, R., Hiley, C. T., Rowan, A. J., Watkins, T. B. K., Wilson, G. A., Birkbak, N. J., Veeriah, S., Van Loo, P., Herrero, J. et al. (2017). Allele-Specific HLA Loss and Immune Escape in Lung Cancer Evolution. Cell 171, 1259-1271.e11.
Meredith, M. M., Liu, K., Darrasse-Jeze, G., Kamphorst, A. O., Schreiber, H. A., Guermonprez, P., Idoyaga, J., Cheong, C., Yao, K. H., Niec, R. E. et al. (2012). Expression of the zinc finger transcription factor zDC (Zbtb46, Btbd4) defines the classical dendritic cell lineage. J Exp Med 209, 1153-1165.
Nastala, C. L., Edington, H. D., McKinney, T. G., Tahara, H., Nalesnik, M. A., Brunda, M. J., Gately, M. K., Wolf, S. F., Schreiber, R. D., and Storkus, W. J. (1994). Recombinant IL-12 administration induces tumor regression in association with IFN-gamma production. J Immunol 153, 1697-1706.
Ngiow, S. F., Young, A., Blake, S. J., Hill, G. R., Yagita, H., Teng, M. W., Korman, A. J., and Smyth, M. J. (2016). Agonistic CD40 mAb-Driven IL12 Reverses Resistance to Anti-PD1 in a T-cell-Rich Tumor. Cancer Res 76, 6266-6277.
Nirschl, C. J., Suárez-Fariñas, M., Izar, B., Prakadan, S., Dannenfelser, R., Tirosh, I., Liu, Y., Zhu, Q., Devi, K. S. P., Carroll, S. L. et al. (2017). IFNγ-Dependent Tissue-Immune Homeostasis Is Co-opted in the Tumor Microenvironment. Cell 170, 127-141.e15.
Paley, M. A., Kroy, D. C., Odorizzi, P. M., Johnnidis, J. B., Dolfi, D. V., Barnett, B. E., Bikoff, E. K., Robertson, E. J., Lauer, G. M., Reiner, S. L. et al. (2012). Progenitor and terminal subsets of CD8+ T cells cooperate to contain chronic viral infection. Science 338, 1220-1225.
Pittet, M. J., Garris, C. S., Arlauckas, S. P., and Weissleder, R. (2018). Recording the wild lives of immune cells. Sci Immunol 3, eaaq0491.
Reinhardt, R. L., Liang, H.-E., Bao, K., Price, A. E., Mohrs, M., Kelly, B. L., and Locksley, R. M. (2015). A novel model for IFN-γ-mediated autoinflammatory syndromes. The Journal of Immunology 194, 2358-2368.
Reinhardt, R. L., Hong, S., Kang, S.-J., Wang, Z.-e., and Locksley, R. M. (2006). Visualization of IL-12/23p40 In Vivo Reveals Immunostimulatory Dendritic Cell Migrants that Promote Thl Differentiation. The Journal of Immunology 177, 1618-1627.
Riaz, N., Havel, J. J., Makarov, V., Desrichard, A., Urba, W. J., Sims, J. S., Hodi, F. S., Martin-Algarra, S., Mandal, R., and Sharfman, W. H. (2017). Tumor and microenvironment evolution during immunotherapy with nivolumab. Cell 171, 934-949. e15.
Rizvi, N. A., Hellmann, M. D., Snyder, A., Kvistborg, P., Makarov, V., Havel, J. J., Lee, W., Yuan, J., Wong, P., Ho, T. S. et al. (2015). Cancer immunology. Mutational landscape determines sensitivity to PD-1 blockade in non-small cell lung cancer. Science 348, 124-128.
Rooney, M. S., Shukla, S. A., Wu, C. J., Getz, G., and Hacohen, N. (2015). Molecular and genetic properties of tumors associated with local immune cytolytic activity. Cell 160, 48-61.
Routy, B., Le Chatelier, E., Derosa, L., Duong, C. P. M., Alou, M. T., Daillère, R., Fluckiger, A., Messaoudene, M., Rauber, C., Roberti, M. P. et al. (2018). Gut microbiome influences efficacy of PD-1-based immunotherapy against epithelial tumors. Science 359, 91-97.
Ruffell, B., Chang-Strachan, D., Chan, V., Rosenbusch, A., Ho, C. M., Pryer, N., Daniel, D., Hwang, E. S., Rugo, H. S., and Coussens, L. M. (2014). Macrophage IL-10 blocks CD8+ T cell-dependent responses to chemotherapy by suppressing IL-12 expression in intratumoral dendritic cells. Cancer Cell 26, 623-637.
Salmon, H., Idoyaga, J., Rahman, A., Leboeuf, M., Remark, R., Jordan, S., Casanova-Acebes, M., Khudoynazarova, M., Agudo, J., Tung, N. et al. (2016). Expansion and Activation of CD103(+) Dendritic Cell Progenitors at the Tumor Site Enhances Tumor Responses to Therapeutic PD-L1 and BRAF Inhibition. Immunity 44, 924-938.
Satija, R., Farrell, J. A., Gennert, D., Schier, A. F., and Regev, A. (2015). Spatial reconstruction of single-cell gene expression data. Nat Biotechnol 33, 495-502.
Schlitzer, A., and Ginhoux, F. (2014). Organization of the mouse and human DC network. Curr Opin Immunol 26, 90-99.
Schreiber, R. D., Old, L. J., and Smyth, M. J. (2011). Cancer immunoediting: integrating immunity's roles in cancer suppression and promotion. Science 331, 1565-1570.
Slifka, M. K., Rodriguez, F., and Whitton, J. L. (1999). Rapid on/off cycling of cytokine production by virus-specific CD8+ T cells. Nature 401, 76-79.
Spranger, S., Bao, R., and Gajewski, T. F. (2015). Melanoma-intrinsic β-catenin signalling prevents anti-tumour immunity. Nature 523, 231-235.
Spranger, S., Koblish, H. K., Horton, B., Scherle, P. A., Newton, R., and Gajewski, T. F. (2014). Mechanism of tumor rejection with doublets of CTLA-4, PD-1/PD-L1, or IDO blockade involves restored IL-2 production and proliferation of CD8+ T cells directly within the tumor microenvironment. Journal for immunotherapy of cancer 2, 1-14.
Szabo, S. J., Kim, S. T., Costa, G. L., Zhang, X., Fathman, C. G., and Glimcher, L. H. (2000). A novel transcription factor, T-bet, directs Thl lineage commitment. Cell 100, 655-669.
Takemoto, N., Intlekofer, A. M., Northrup, J. T., Wherry, E. J., and Reiner, S. L. (2006). Cutting Edge: IL-12 inversely regulates T-bet and eomesodermin expression during pathogen-induced CD8+ T cell differentiation. J Immunol/77, 7515-7519.
Thurber, G. M., Yang, K. S., Reiner, T., Kohler, R. H., Sorger, P., Mitchison, T., and Weissleder, R. (2013). Single-cell and subcellular pharmacokinetic imaging allows insight into drug action in vivo. Nat Commun 4, 1504.1-10.
Topalian, S. L., Hodi, F. S., Brahmer, J. R., Gettinger, S. N., Smith, D. C., McDermott, D. F., Powderly, J. D., Carvajal, R. D., Sosman, J. A., and Atkins, M. B. (2012). Safety, activity, and immune correlates of anti-PD-1 antibody in cancer. New England Journal of Medicine 366, 2443-2454.
Weinreb, C., Wolock, S., and Klein, A. (2017). SPRING: a kinetic interface for visualizing high dimensional single-cell expression data. Bioinformatics 34, 1246-1248.
Weissleder, R., Nahrendorf, M., and Pittet, M. J. (2014). Imaging macrophages with nanoparticles. Nat Mater 13, 125-138.
Wherry, E. J. (2011). T cell exhaustion. Nature Immunology 12, 492-499.
Yan, J., Smyth, M. J., and Teng, M. W. L. (2017). Interleukin (IL)-12 and IL-23 and Their Conflicting Roles in Cancer. Cold Spring Harb Perspect Biol 10, 7. 1-18.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject with cancer, comprising administering an inhibitor of the non-canonical NFkB pathway and a checkpoint inhibitor.

2. The method of claim 1, wherein the subject has melanoma, brain cancer, or colorectal cancer.

3. The method of claim 1, wherein the subject is a human.

4. The method of claim 1, wherein the checkpoint inhibitor is an antibody.

5. The method of claim 4, wherein the antibody is anti-PD1 or anti-PDL1.

6. The method of claim 1, wherein the inhibitor of the non-canonical NFkB pathway is a NIK inhibitor.

7. The method of claim 6, wherein the NIK inhibitor is selected from the group consisting of alkynyl alcohols; 6-membered heteroaromatic substituted cyanoindoline derivatives; pyrazoloisoquinoline derivatives; 6-azaindole aminopyrimidine derivatives; pyrazoloisoquinoline derivatives; sulfapyridine; propranolol; tricyclic NF-κB inducing kinase inhibitors; 4H-isoquinoline-1,3-dione and 2,7-naphthydrine-1,3,6,8-tetrone; N-Acetyl-3-aminopyrazoles; NIK-SMI1 ((R)-6-(3-((3-hydroxy-1-methyl-2-oxopyrrolidin-3-yl)ethynyl)phenyl)-4-methoxypicolinamide), AM-0216 ((R)-4-(1-(2-aminopyrimidin-4-yl)indolin-6-yl)-2-(thiazol-2-yl)but-3-yn-2-ol), AM-0561 ((R)-4-(3-(2-amino-5-chloropyrimidin-4-yl)imidazo[1,2-a]pyridin-6-yl)-2-(thiazol-2-yl)but-3-yn-2-ol), or Amgen16 (1-((1-(2-amino-5-chloropyrimidin-4-yl)indolin-6-yl)ethynyl)cyclopentan-1-ol).

8. The method of claim 1, wherein the inhibitor of the non-canonical NFkB pathway and the checkpoint inhibitor are administered in a single composition.

9-16. (canceled)

17. The method of claim 2, wherein the brain cancer is glioblastoma (GBM).
